(19) United States
(12) Patent Application Publication
(10) Pub. No.: US 2026/0341505 A1
(43) Pub. Date: Oct. 29, 2026

(54) Method for Improving the Diagnosis of Rare Diseases Using Electronic Health Records Data and Systems for Same

(71) Applicant: The Regents of the University of California, Oakland, CA (US)

(72) Inventors: Vivek Rudrapatna, San Francisco, CA (US); Balu Bhasuran, San Francisco, CA (US)

(21) Appl. No.: 19/107,462

(22) PCT Filed: Aug. 30, 2023
(86) PCT No.: PCT/US2023/031492

§ 371 Date: Feb. 27, 2025

Related U.S. Application Data
(60) Provisional application No. 63/374,488, filed on Sep. 2, 2022.

(51) Int. Cl. G16H 50/20; G06N 20/00; G16H 10/60

(52) U.S. Cl. CPC G16H 50/20; G06N 20/00; G16H 10/60

(57) ABSTRACT

Methods and systems for improving the diagnosis of rare diseases using electronic health records data include training a model to estimate the presence of a disease in a subject comprising: obtaining a plurality of health records comprising health information about individuals, modifying each health record of the plurality of health records to exclude certain health information, identifying potential indicators of a disease based on publicly available databases, where the disease is a rare disease (e.g., a disease that occurs in approximately one in 100,000 individuals), and the potential indicators comprise health information present in the health records and training the machine learning model using the plurality of health records and based at least in part on the results of evaluating the accuracy of the machine learning model to recommend a referral for evaluating the presence of a disease in one or more subjects.

### CROSS REFERENCE TO APPLICATIONS

This application claims the benefit of U.S. Provisional Application Ser. No. 63/374,488, filed on Sep. 2, 2023, which application is incorporated by reference herein.

### INTRODUCTION

The paradox of rare diseases is that they collectively explain up to 5% of the disease burden seen in a typical practice, yet are individually uncommon, for example occurring with a frequency of less than 1 in 10,000 or less than 1 in 100,000. Diagnosing rare diseases requires clinical expertise, which typically is in limited supply, and significant diagnostic delays are common. Many attempts have been made by a variety of data science researchers to develop methods for improving the diagnosis of rare diseases. However, such methods have suffered from a variety of limitations, including, for example: (1) failure to account for known confounding biases in observational datasets (i.e., non-random decision to pursue a specific diagnostic evaluation); (2) improper handling of time (i.e., incorporating future, post-diagnostic data as predictors of the diagnosis); as well as (3) suboptimal methods of training predictive models (i.e., not using publicly-available knowledge to improve learning efficiency). In addition, use of electronic health records data in connection with predictive modeling remains challenging in part due to: machine learning typically needs “big data,” with a good balance of cases and controls, which may be problematic for rare disease; and most of the useful electronic health records data has been captured in a free-text format, meaning it is not structured, not immediately useful for research goals such as predicting future diagnoses.

### SUMMARY

Thus, there is a need for improved and useful methods and systems for improving the diagnosis of rare diseases. This invention provides such new and useful methods and systems, addressing the limitations mentioned above. To accomplish this, the invention leverages electronic health record data as well as machine learning techniques to estimate the presence of a disease, such as a rare disease, in a subject and/or recommend a referral for a subject for further evaluation. Embodiments of the present invention are capable of accurately identifying exemplary rare diseases in confirmed patients from more than one medical center and also recognizing when to abstain from making a diagnostic prediction. Embodiments of the present invention draw from several disciplines to overcome technical challenges and address unmet clinical needs, including: natural language processing for transforming notes into analyzable information; informatics for accessing already structured data in electronic health records databases and complement info from notes; machine learning for training algorithms to recognize cases/controls using examples; as well as knowledge graphs for using codified knowledge about rare diseases, such as, for example, AHP, to direct the learning process and help algorithms become smarter learners in the face of limited training examples.

Methods and systems for improving the diagnosis of rare diseases using electronic health records data are provided. Aspects of the present invention include methods of training a model to estimate the presence of a disease in a subject comprising: obtaining a plurality of health records comprising health information about individuals, modifying each health record of the plurality of health records to exclude certain health information, identifying potential indicators of a disease based on publicly available databases, where the disease is a rare disease (e.g., a disease that occurs in approximately one in 100,000 individuals), and the potential indicators comprise health information present in the health records, training a machine learning model to estimate the presence of the disease based on the potential indicators of the disease using a first subset of the modified health records, evaluating the accuracy of estimates of the presence of the disease based on the potential indicators of the disease made by the machine learning model using a second subset of the modified health records, and further training the machine learning model using the plurality of health records and based at least in part on the results of evaluating the accuracy of the machine learning model. Aspects of the present invention further include methods of inferring the presence of a disease in one or more subjects; methods of clinically evaluating the presence of a disease in one or more subjects; methods of training a model to recommend a referral for evaluating the presence of a disease in one or more subjects; as well as methods of recommending a referral for evaluating the presence of a disease in one or more subjects. Also provided are systems for performing the methods described herein as well as non-transitory computer readable storage media.

The methods and systems find use in a variety of different applications, e.g., diagnosing subjects with rare diseases (e.g., diseases occurring with a frequency of less than 1 in 10,000 or less than 1 in 100,000), such as, for example, subjects with acute hepatic porphyria (AHP), a group of four heritable metabolic diseases of the heme biosynthesis pathway. All four diseases present with episodic, severe, neurovisceral attacks which are characterized by abnormal accumulation of the heme precursors delta-aminolevulinic acid (ALA) and porphobilinogen (PBG). ALA in particular is believed to be a neurotoxin, which causes potentially life-threatening injury to the nervous system and other organs. Further details are provided in: Puy H, Gouya L, Deybach J C. Porphyrias. The Lancet. 2010; 375(9718):924-937. doi:10.1016/S0140-6736(09)61925-5, the disclosure of which is incorporated herein in its entirety. AHP patients are also at risk for chronic comorbidities including liver disease, chronic kidney disease, and chronic neuropathy. This is believed to be due to long-term elevations of ALA and PBG. Further details are provided in: Balwani M, Wang B, Anderson K E, et al. Acute hepatic porphyrias: Recommendations for evaluation and long-term management. Hepatology. 2017;66(4):1314-1322. doi:10.1002/hep.29313, the disclosure of which is incorporated herein in its entirety. AHP is associated with significant diagnostic delays, as much as 15 years in one study due to: it's rarity, with prevalence of symptomatic AHP occurring at approximately 1 in 100,000 individuals; it's easy to misdiagnose with cardinal symptoms including acute abdominal pain, particularly in women; and local clinical expertise is typically limited. Further details are provided in: Bonkovsky H L, Maddukuri V C, Yazici C, Anderson K E, Bissell D M, Bloomer J R, Phillips J D, Naik H, Peter I, Baillargeon G, Bossi K, Gandolfo L, Light C, Bishop D, Desnick R J. Acute porphyrias in the USA: features of 108 subjects from porphyrias consortium. Am J Med. 2014 Dec;127(12):1233-41. doi:10.1016/j.amjmed.2014.06.036. Epub 2014 Jul. 10. PMID: 25016127; PMCID: PMC4563803, the disclosure of which is incorporated herein in its entirety. The typical treatment of AHP involves the intravenous administration of hemin during the acute phase. However, the successful development and approval of Givosiran, a small interfering RNA product directed against systemic ALAS1, now provides an effective prophylactic option for AHP patients with chronically elevated ALA and recurrent attacks. An important next step is to reduce diagnostic delays in potentially undiagnosed patients.

A study of 108 acute hepatic porphyria cases in the United States found that the average delay from the onset of symptoms to diagnosis was 15 years.

Further details are provided in: Bonkovsky H L, Maddukuri V C, Yazici C, Anderson K E, Bissell D M, Bloomer J R, Phillips J D, Naik H, Peter I, Baillargeon G, Bossi K, Gandolfo L, Light C, Bishop D, Desnick R J. Acute porphyrias in the USA: features of 108 subjects from porphyrias consortium. Am J Med. 2014 December;127(12):1233-41. doi:10.1016/j.amjmed.2014.06.036. Epub 2014 Jul. 10. PMID: 25016127; PMCID: PMC4563803, the disclosure of which is incorporated herein in its entirety. Health care providers do not consider AHP in their differential diagnosis because it is thought to be very rare and there is a lack of familiarity with symptoms and appropriate diagnostic tests. The generally cited estimate of symptomatic AHP is approximately 1 per 100,000 individuals in the general population. However, recent population studies have shown that the genetic carrier state is much higher than previously thought of, such as 1 per 1,500 individuals. Further details are provided in: Lenglet H, Schmitt C, Grange T, et al. From a dominant to an oligogenic model of inheritance with environmental modifiers in acute intermittent porphyria. Human Molecular Genetics. 2018; 27(7): 1164-1173. doi:10.1093/hmg/ddy030, the disclosure of which is incorporated herein in its entirety. Still Further details are provided in: Chen B, Solis-Villa C, Hakenberg J, et al. Acute Intermittent Porphyria: Predicted Pathogenicity of HMBS Variants Indicates Extremely Low Penetrance of the Autosomal Dominant Disease. Human Mutation. 2016; 37(11): 1215-1222. doi:10.1002/humu.23067, the disclosure of which is incorporated herein in its entirety. About half of all symptomatic patients with AHP had one attack in the last year and about 10-20% had hemin use recently. Further details are provided in: Acute Hepatic Porphyrias: Review and Recent Progress—Wang—2019 Hepatology Communications—Wiley Online Library. Accessed Jun. 15, 2022. https://aasldpubs.onlinelibrary.wiley.com/doi/full/10.1002/hep4.1297, the disclosure of which is incorporated herein in its entirety. Furthermore, a subset of AHP patients is known to have chronically elevated ALA and PBG levels without clear clinical history of acute attacks. This population of asymptomatic high excretors (ASHE) is still at risk for some chronic comorbidities, including liver diseases such as cirrhosis and liver cancer. Further details are provided in: Balwani M, Wang B, Anderson K E, et al. Acute hepatic porphyrias: Recommendations for evaluation and long-term management. Hepatology. 2017;66(4):1314-1322. doi:10.1002/hep.29313, the disclosure of which is incorporated herein in its entirety.

Electronic health records (EHRs) containing structured and unstructured healthcare data of individuals and population provides an immense opportunity to generate disease diagnostic prediction models7. Further details are provided in: Rudrapatna V A, Butte A J. Opportunities and challenges in using real-world data for health care. J Clin Invest. 2020;130(2):565-574. doi:10.1172/JCI129197, the disclosure of which is incorporated herein in its entirety. However, due to complexity and diversity, data missingness, and richer knowledge availability through free text such as clinical notes makes EHR-based model generation a challenging task. Further details are provided in: Pathak J, Kho A N, Denny J C. Electronic health records-driven phenotyping: challenges, recent advances, and perspectives. Journal of the American Medical Informatics Association. 2013;20(e2):e206-e211.doi:10.1136/amiajnl-2013-002428; Hripcsak G, Albers D J. Next-generation phenotyping of electronic health records. Journal of the American Medical Informatics Association. 2013;20(1):117-121. doi:10.1136/amiajnl-2012-001145; Rajkomar A, Oren E, Chen K, et al. Scalable and accurate deep learning with electronic health records. npj Digital Med. 2018;1(1):1-10. doi:10.1038/s41746-018-0029-1, the disclosures of each of which are incorporated herein in their entirety. Specifically, in the case of rare diseases, unstructured data explicitly mentioned in notes extracted using natural language processing (NLP) technology can be complementary to structured patient data for machine learning algorithms. Further details are provided in: Further details regarding such approach are provided in: Lo Barco T, Kuchenbuch M, Garcelon N, Neuraz A, Nabbout R. Improving early diagnosis of rare diseases using Natural Language Processing in unstructured medical records: an illustration from Dravet syndrome. Orphanet Journal of Rare Diseases. 2021; 16(1):309. doi:10.1186/s13023-021-01936-9, the disclosure of which is herein incorporated in its entirety. Systematic analysis of EHR data covering the entire patient health history through signs, symptoms, medical diagnosis, and evaluation can facilitate reducing diagnostic delays in AHP.

Embodiments of the methods, systems and non-transitory computer readable media described herein provide advantages, in part, because in some cases they enable: use of clinical notes as health information, feature selection using external biomedical knowledge sources (e.g., SemMedDB and GARD, as described below), data restriction on health information (i.e., excluding certain aspects of electronic health records based on dates of clinically relevant events), two-stage modeling approach (i.e., training both referral and diagnosis models and, for example, first applying a referral model to a select population prior to applying a diagnosis model to a subset of the patient population determined in part based on the results of applying the referral model) as well as model evaluation using baseline, autoML and deep learning approaches.

### BRIEF DESCRIPTION OF THE FIGURES

The invention may be best understood from the following detailed description when read in conjunction with the accompanying drawings. Included in the drawings are the following figures:

FIG. 1 illustrates a flow diagram for training and applying a model for estimating the presence of a rare disease and training and applying a model for recommending a referral for a rare disease according to embodiments of the present invention.

FIG. 2 depicts a causal model diagram that illustrates an exemplary patient trajectory involving selection bias leading to a rare disease diagnosis.

FIG. 3 depicts a flow diagram corresponding to an embodiment of a method of the present invention in the context of subject health records, i.e., sequential patient history of health events recorded in a health record.

FIG. 4A illustrates application of an embodiment of a method according to the present invention. FIG. 4B presents probability distribution results from applying embodiments of referral and diagnosis models discussed in connection with FIG. 4A.

FIG. 5 presents a rank ordered list of AHP disease specific features identified in connection with applying the embodiment presented in FIG. 4A.

FIGS. 6A-6D present results and analysis in connection with applying the embodiment of the present invention discussed in connection with FIG. 4A.

FIGS. 7A-D present results and analysis in connection with applying the embodiment of the present invention discussed in connection with FIG. 4A.

FIGS. 8A-B present results and analysis in connection with applying the embodiment of the present invention discussed in connection with FIG. 4A.

FIG. 9 presents results and analysis in connection with applying the embodiment of the present invention discussed in connection with FIG. 4A.

### DETAILED DESCRIPTION

Aspects of the present invention include methods of training a model to estimate the presence of a disease in a subject comprising: obtaining a plurality of health records comprising health information about individuals, modifying each health record of the plurality of health records to exclude certain health information, identifying potential indicators of a disease based on publicly available databases, where the disease is a rare disease (e.g., a disease that occurs in approximately one in 100,000 individuals), and the potential indicators comprise health information present in the health records, training a machine learning model to estimate the presence of the disease based on the potential indicators of the disease using a first subset of the modified health records, evaluating the accuracy of estimates of the presence of the disease based on the potential indicators of the disease made by the machine learning model using a second subset of the modified health records, and further training the machine learning model using the plurality of health records and based at least in part on the results of evaluating the accuracy of the machine learning model. Aspects of the present invention further include methods of inferring the presence of a disease in one or more subjects; methods of clinically evaluating the presence of a disease in one or more subjects; methods of training a model to recommend a referral for evaluating the presence of a disease in one or more subjects; as well as methods of recommending a referral for evaluating the presence of a disease in one or more subjects. Also provided are systems for performing the methods described herein as well as non-transitory computer readable storage media.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the system and method may be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

As summarized above, the present disclosure provides methods and systems for improving the diagnosis of rare diseases using electronic health records. By “rare disease,” it is meant any disease capable of detection and diagnosis. In some cases, “rare disease” refers to diseases with a relatively rare population of individuals, i.e., subjects, affected by the disease. For example, in some cases, rare diseases are diseases that occur in approximately 1 in 200,000 individuals or 1 in 100,000 individuals or 1 in 10,000 individuals or 1 in 1,000 individuals. In other cases, rare diseases include diseases that affect fewer than 200,000 people in the United States, for example. In some cases, disease prevalence, meaning the number of people living with the disease at a given moment is used to define rare diseases. In other cases, disease incidence, meaning the number of new diagnoses in a given year, is used to define a rare disease.

Examples of rare diseases include but are not limited to those described in publicly available resources such as The United States Genetic and Rare Diseases Information Center, available at: https://rarediseases.info.nih.gov/about, or the National Organization for Rare Disorders (NORD), available at: https://rarediseases.org/, or orphanet, available at: https://www.orpha.net/consor/cgi-bin/index.php. Each such resource is incorporated herein by reference in their entirety. Other examples or rare diseases of interest include, for example, acute hepatic porphyria (AHP), a heritable metabolic disorder of the heme biosynthesis pathway. Still other examples of rare diseases of interest include factor XIII deficiency, Hutchinson-Gilford progeria syndrome, Barth syndrome (BTHS), nephrogenic diabetes insipidus, congenital generalized lipodystrophy (also known as Berardinelli-Seip lipodystrophy), fibrolamellar hepatocellular carcinoma (FHCC), autoimmune polyendocrine syndrome type 1 (APS-1), cerebral creatine deficiency syndromes, cyclic neutropenia, goblet cell carcinoid, ablepharon-macrostomia syndrome (AMS), Alexander disease, Baller-Gerold syndrome (BGS), Bernard-Soulier syndrome (BSS), Caroli disease, Cleidocranial dysostosis (CCD), cold agglutinin disease (CAD), congenital afibrinogenemia, cutaneous T cell lymphoma (CTCL), cutis laxa, factor XI or plasma thromboplastin antecedent, factor XII deficiency, familial partial lipodystrophy, fatal insomnia, glycogen storage disease type VI (GSD VI), hypothalamic disease, keratosis follicularis spinulosa decalvans, Miller syndrome, opsoclonus myoclonus syndrome (OMS), paroxysmal nocturnal hemoglobinuria (PNH) or Crigler-Najjar syndrome.

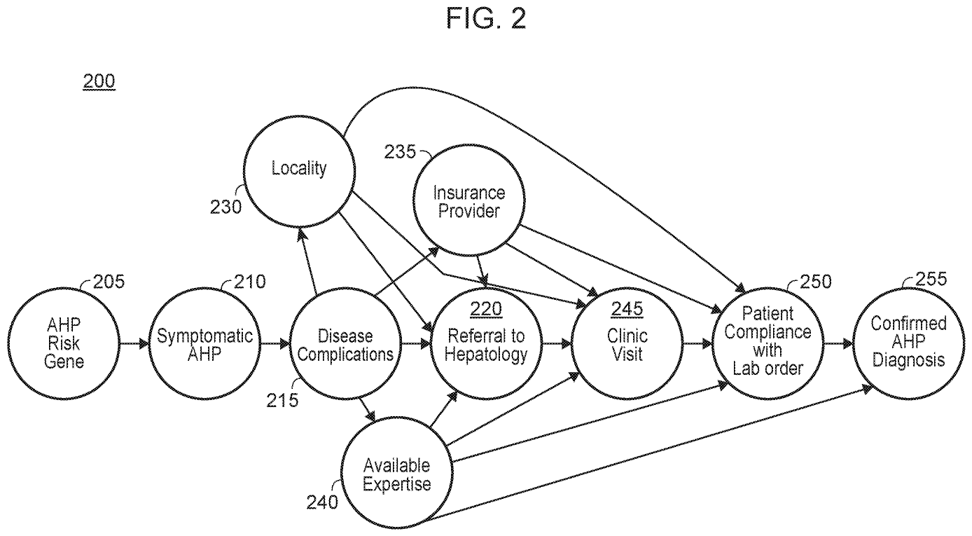

By estimating the presence of a disease in a subject, it is meant estimating a likelihood that a subject would be diagnosed with the rare disease using techniques available in the prevailing standard of care. By determining whether a referral is warranted, it is meant estimating a likelihood of whether a referral of a subject to a specialist for evaluation of whether a subject can be diagnosed with a rare disease is called for in view of the data available in a health record. In some embodiments, by determining whether a referral is warranted, it is meant estimating the likelihood of a referral to a specialist for clinical evaluation a rare disease. (For further details on the clinical stages of patient treatment that correspond to each of estimating a likelihood that a subject would be diagnosed and estimating a likelihood of whether a referral of a subject to a specialist is warranted, see the discussion below in connection with FIG. 2, in particular, steps 220 and 255 of the model 200 in FIG. 2).

In embodiments, the individual, i.e., the subject, about which a diagnosis or referral may be made according to the present invention, is generally a human subject and may be male or female of any age and with no specific medical history or history of disease or family history of disease.

### Methods for Improving the Diagnosis of Rare Diseases Using Electronic Health Records Data

Aspects of the present disclosure include methods for training and applying a model for estimating the presence of a rare disease and training and applying a model for recommending a referral for a rare disease according to embodiments of the present invention. In particular, the present disclosure includes methods of training a model to estimate the presence of a disease in a subject comprising: obtaining a plurality of health records comprising health information about individuals, modifying each health record of the plurality of health records to exclude certain health information, identifying potential indicators of a disease based on publicly available databases, wherein the disease is a rare disease (e.g., a disease that occurs in approximately one in 100,000 individuals), and the potential indicators comprise health information present in the health records, training a machine learning model to estimate the presence of the disease based on the potential indicators of the disease using a first subset of the modified health records, evaluating the accuracy of estimates of the presence of the disease based on the potential indicators of the disease made by the machine learning model using a second subset of the modified health records, and further training the machine learning model using the plurality of health records and based at least in part on the results of evaluating the accuracy of the machine learning model.

The present disclosure further includes methods of inferring the presence of a disease in a subject comprising: obtaining a health record comprising health information about a subject, applying a model trained according to the methods disclosed herein to the health record of the subject to estimate the presence in a disease in the subject, and estimating the presence of the disease in the subject based on the results of applying the model.

The present disclosure further includes methods of clinically evaluating the presence of a disease in a subject comprising: estimating the presence of a disease in a subject according to the methods described herein, wherein the estimate of the presence of the disease comprises a confidence score, and clinically evaluating the presence of the disease in the subject, in the event the confidence score exceeds a predetermined threshold.

The present disclosure further includes methods of estimating the presence of a disease in a plurality of subjects comprising: obtaining health records comprising health information about a plurality of subjects, applying a model trained according to the methods disclosed herein to the health records to estimate the presence in a disease in each subject of the plurality of subjects, and inferring the presence of the disease in each subject of the plurality of subjects based on the results of applying the model.

The present disclosure further includes methods of clinically evaluating the presence of a disease in a plurality of subjects comprising: estimating the presence of a disease in a plurality of subjects according to the methods described herein, wherein each estimate of the presence of the disease in each subject of the plurality of subjects comprises a confidence score, and clinically evaluating the presence of the disease in each subject, in the event the confidence score for such subject exceeds a predetermined threshold.

The present disclosure further includes methods of training a model to recommend a referral for evaluating the presence of a disease in a subject comprising: obtaining a plurality of health records comprising health information about individuals, modifying each health record of the plurality of health records to exclude certain health information, identifying potential indicators of a disease based on publicly available databases, wherein the disease is a rare disease (e.g., a disease that occurs in approximately one in 100,000 individuals), and the potential indicators comprise health information present in the health records, training a machine learning model to recommend a referral for evaluating the presence of a disease based on the potential indicators of the disease using a first subset of the modified health records, evaluating the accuracy of the machine learning model based on the potential indicators of the disease using a second subset of the modified health records, and further training the machine learning model using the plurality of health records and based at least in part on the results of evaluating the accuracy of the machine learning model.

The present disclosure further includes methods of recommending a referral for evaluating the presence of a disease in a subject comprising: obtaining a health record comprising health information about a subject, applying a model trained according to the methods disclosed herein to the health record of the subject to recommend a referral for evaluating the presence of a disease in the subject, and recommending a referral for evaluating the presence of a disease in the subject based on the results of applying the model.

The present disclosure further includes methods of estimating the presence of a disease in a subject comprising: determining whether a referral for evaluating the presence of a disease in a subject is recommended according to the methods disclosed herein, wherein the recommendation for a referral comprises a confidence score, and applying a model trained according to the methods disclosed herein to a health record of the subject to estimate the presence in a disease in the subject, in the event the confidence score exceeds a predetermined threshold, and estimating the presence of the disease in the subject based on the results of applying the model.

The present disclosure further includes methods of clinically evaluating the presence of a disease in a subject comprising: estimating the presence of a disease in a subject according to the methods described herein, wherein the estimate of the presence of the disease comprises a confidence score, and clinically evaluating the presence of the disease, in the event the confidence score exceeds a predetermined threshold.

The present disclosure further includes methods of recommending a referral for evaluating the presence of a disease in a plurality of subjects comprising: obtaining health records comprising health information about a plurality of subjects, applying a model trained according to the methods described herein to the health records to recommend a referral for evaluating the presence of a disease in each subject of the plurality of subjects, and recommending a referral for evaluating the presence of a disease in each subject of the plurality of subjects based on the results of applying the model.

The present disclosure further includes methods of estimating the presence of a disease in a plurality of subjects comprising: determining whether a referral for evaluating the presence of a disease in each subject of a plurality of subjects is recommended according to the methods described herein, wherein the recommendation for a referral comprises a confidence score, applying a model trained according to the methods disclosed herein to health records of each subject of the plurality of subjects to estimate the presence of a disease of each subject of the plurality of subjects, in the event the confidence score exceeds a predetermined threshold, and estimating the presence of the disease in each subject of the plurality of subjects based on the results of applying the model.

The present disclosure further includes methods of clinically evaluating the presence of a disease in a plurality of subjects comprising: estimating the presence of a disease in a plurality of subjects according to the methods disclosed herein, wherein each estimate of the presence of the disease comprises a confidence score, and clinically evaluating the presence of the disease for each subject of the plurality of subjects, in the event the confidence score exceeds a predetermined threshold.

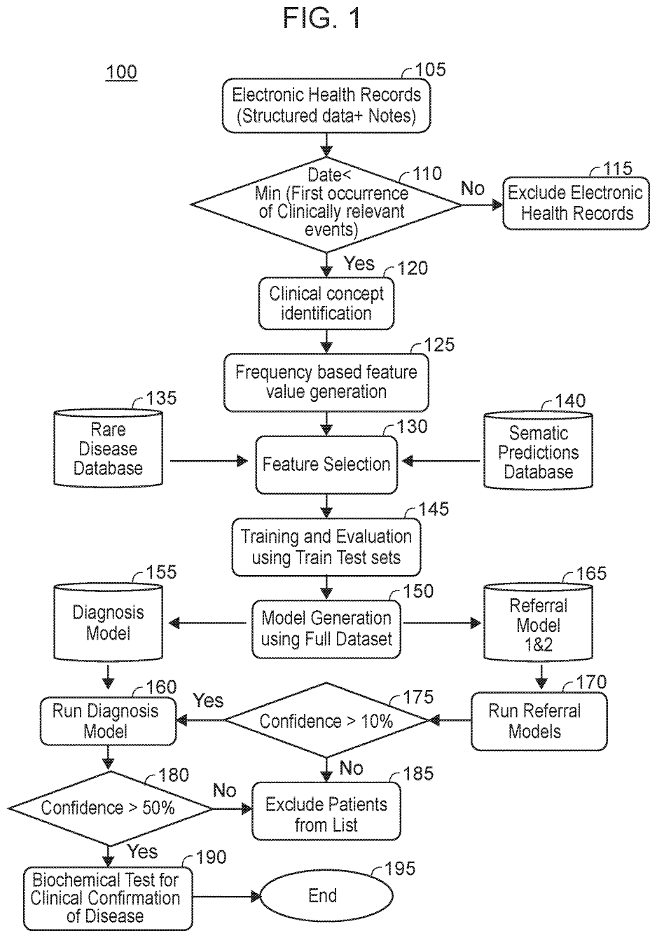

FIG. 1 illustrates a flow diagram 100 for training and applying a model for estimating the presence of a rare disease and training and applying a model for recommending a referral for a rare disease according to embodiments of the present invention. The embodiment of the present invention depicted in FIG. 1 relates to diagnosing or referring a subject for the rare disease Acute Hepatic Porphyria (AHP), a heritable metabolic disorder of the heme biosynthesis pathway. Flow diagram 100 is an exemplary embodiment of the present invention provided for illustrative purposes; the disease acute hepatic porphyria (AHP) is also provided for illustrative purposes, and it is to be understood that the present invention is not limited to methods solely related to acute hepatic porphyria (AHP).

Flow diagram 100 starts at step 105. At step 105, subject (i.e., patient) health records are identified. Health records of interest comprise electronic health records with health information about one or more subjects, including both structured and unstructured electronic health records data. Health records of interest may be generated by a subject's healthcare team, such as doctors, including general care practitioners or specialists or other medical professionals that encounter the subject. Health records of interest may be obtained from medical centers, such as major medical centers including, for example, UCSF or UCLA medical centers. Health records of interest may be combined records from more than one institution or medical care provider. Health records of interest may include clinical notes. Further details regarding health records of interest are provided in: Rudrapatna VA, Butte AJ. Opportunities and challenges in using real-world data for health care. J Clin Invest. 2020;130(2):565-574. doi:10.1172/JCI129197; Pathak J, Kho AN, Denny J C. Electronic health records-driven phenotyping: challenges, recent advances, and perspectives. Journal of the American Medical Informatics Association. 2013; 20(e2):e206-e211. doi:10.1136/amiajnl-2013-002428; Hripcsak G, Albers D J. Next-generation phenotyping of electronic health records. Journal of the American Medical Informatics Association. 2013;20(1):117-121. doi:10.1136/amiajnl-2012-001145; Rajkomar A, Oren E, Chen K, et al. Scalable and accurate deep learning with electronic health records. npj Digital Med. 2018;1(1):1-10. doi:10.1038/s41746-018-0029-1, the disclosures of each of which are incorporated herein in their entirety. In embodiments, health record data may cover the entire patient health history through signs, symptoms, medical diagnosis and evaluation or aspects or subsets thereof. That is, health record data may be incomplete with respect to certain aspects of a subject's medical history. Health records of interest may be available from, for example, Epic, including offerings such as MyChart.

By “structured health record,” it is meant that a subject's health data is presented in a structured format, such as a database format, with, for example, labeled or tagged numerical results, or otherwise organized in a regular or structured format for ease of sorting, searching and analysis. By “unstructured health record,” it is meant a subject's health data that is not presented in a structured format, such as notes, descriptions, drawings or other markings that are not, for example, numerical or responsive to a specific, structured database field. In embodiments, unstructured health record data comprises information in a free-text format. Unstructured health record data present in notes associated with a health record may be extracted using natural language processing (NLP) technology. Further details regarding such approach are provided in: Lo Barco T, Kuchenbuch M, Garcelon N, Neuraz A, Nabbout R. Improving early diagnosis of rare diseases using Natural Language Processing in unstructured medical records: an illustration from Dravet syndrome. Orphanet Journal of Rare Diseases. 2021;16(1):309. doi:10.1186/s13023-021-01936-9, the disclosure of which is herein incorporated in its entirety. In embodiments, unstructured health record data can be complementary to structured health record data in connection with training machine learning algorithms. Embodiments of the present invention may utilize structured health records or both structured and unstructured health records. After step 105, the process moves to step 110.

At step 110, health records identified at block 105 are analyzed to determine the presence of and, if applicable, the date of, a first occurrence of a clinically relevant event. By “clinically relevant event,” it is meant, for example, a disease code assignment, a negative confirmation of whether the subject has the rare disease of interest, in this case, acute hepatic porphyria (AHP), or whether the subject has visited with a specialist, in this case, a Porphyria clinician. At step 110, in the event health records indicate a clinically relevant event has occurred prior to a specified date, such health records, or aspects thereof, i.e., a subset of the applicable health record, are excluded at step 115. That is, the process moves to step 115 from step 110 if the health record comprises one or more clinically relevant events, as described above. With respect to health records where it was determined that no clinically relevant event occurred prior to a specified date (i.e., health records that were not excluded at step 110), the process moves to step 120.

Step 120 entails clinical concept identification from notes associated with subject health records. That is, a clinical concept is identified and/or generated and/or developed based on the subject's health records filtered at step 110, i.e., the aspects of the subject's health record that were not excluded at step 120 but proceeded to step 120. By “clinical concept identification,” it is meant identification of clinically relevant information present in the electronic health records. After clinical concept identification at step 120, the process moves to step 125.

At step 125, the subject's health records are analyzed using frequency based feature value generation. By “features,” it is meant characteristics of the disease, acute hepatic porphyria (AHP), that may be present in the subject's health records. The terms feature or indicator in the context of disease feature or disease indicator may be used interchangeably throughout this disclosure. For example, in embodiments, feature selection is intended to enable models to learn clinically relevant signs and symptoms, such as, for example, “abdominal pain,” “nausea and vomiting,” “hypertension,” “tremor,” etc. rather than concepts like “PBG,” “ALA,” “hemin,” “hepatology,” etc., which may occur in certain health records but not others, such as case or control health records.

By analysis of the frequency of features present in relevant electronic health records, certain features are identified as potential predictors for estimating the presence of the disease, acute hepatic porphyria (AHP). That is, certain features or characteristics or symptoms or health conditions or health concerns or any other aspect or data present in the health records may be assigned values based on their potential as predictors of disease. In embodiments, natural language based processing is used to identify and analyze information present in health records. Once feature values are assigned based on frequency patterns at step 125, the process moves to step 130.

At step 130, a selection of features, as described above, is further refined, such that certain features or characteristics may be treated as disease specific predictors. Such feature selection is further refined using information from semantic prediction 140 and rare disease prediction 135 databases. That is, at step 130, certain features or characteristics or symptoms or health conditions or health concerns or any other aspect or data present in the health records are selected as relevant features for use in estimating a diagnosis or referral for the disease. At step 130, such disease specific features are determined based in part on (i) the frequency analysis of features identified in step 125, as described above as well as (ii) information from publicly available databases relating to rare disease conditions at step 135 (e.g., the results of querying a rare disease database regarding the disease, acute hepatic porphyria (AHP)) as well as (iii) information from publicly available databases relating to semantic predictions regarding the disease, acute hepatic porphyria (AHP)). In embodiments, rare disease database 135 may comprise any relevant database comprising information about the disease, acute hepatic porphyria (AHP). For example, rare disease database 135 may comprise information regarding findings about the disease, acute hepatic porphyria (AHP), such as expected effects of the presence of the disease in a subject or expected symptoms exhibited in a subject where the disease is present or information about the mechanisms, e.g., biochemical pathways or other physiological aspects or processes, which the disease is expected to disrupt when present in a subject. In embodiments, semantic predictions database 140 may comprise any convenient database comprising information about making predictions related to a disease based on semantic information. In some cases, semantic predictions database 140 comprises a database that facilitates predicting the presence of a disease based on semantic information present in, for example, the health records of subjects identified at step 105. After feature selection of disease specific predictors are determined at step 130, the process moves to step 145.

At step 145, a model is trained and tested (i.e., evaluated) to estimate the presence of, or recommend a referral for, the disease, acute hepatic porphyria (AHP). In embodiments, the model is any convenient model capable of training to estimate the presence of, or recommend a referral for, the disease, acute hepatic porphyria (AHP). In embodiments, the model is a computational model. Any convenient computational model may be applied, such as a statistical model, machine learning model, convolutional neural network or the like. In still other cases, the model comprises an artificial neural network or deep learning network. Training a model refers to configuring, fitting or otherwise preparing a model to make predictions, such as, for example, to estimate the presence of, or recommend a referral for, the disease, acute hepatic porphyria (AHP), and is distinguished from applying a model to make predictions about estimating the presence of, or recommend a referral for, the disease, acute hepatic porphyria (AHP) (i.e., distinct from applying the model to non-training data). With respect to training a model, embodiments of a model are trained using health records of subjects in conjunction with disease specific features identified and selected at step 130. In embodiments, the model is trained using one or more subsets of the health records of subjects, i.e., training data, which may consist of instances of health records corresponding to subjects ultimately known to be diagnosed with the disease, acute hepatic porphyria (AHP), as well as health records corresponding to subjects ultimately known to not have the disease, acute hepatic porphyria (AHP).

In embodiments, training a model using at least a subset of the health records comprises applying an unsupervised learning technique to the model.

Unsupervised learning is a machine learning technique known in the art for training a model to, for example, identify or recognize patterns. Unsupervised learning comprises training a model where pre-assigned labels (e.g., regarding the presence of or a recommendation for a referral for the disease, acute hepatic porphyria (AHP)) are not provided to the model with respect to data used to train the model. That is, in the case of embodiments of the invention, no labels indicating whether training health records correspond to health records of subjects known to have (or known not to have) the disease or known to need (or known not to need) a referral for evaluation of the disease are provided in connection with training the model. As a result, applying unsupervised learning to train a model entails the model itself discovering patterns among the training data.

In other embodiments, training a model using at least a subset of the health records of subjects in conjunction with the disease specific features identified and selected above comprises applying a semi-supervised learning technique to the model. Semi-supervised learning is a machine learning technique known in the art for training a model to, for example, identify or recognize patterns. Semi-supervised learning comprises training a model using both labeled and unlabeled training data.

In some embodiments, training a model using at least a subset of the health records of subjects in conjunction with the disease specific features identified and selected above comprises applying a supervised learning technique to the model. Supervised learning is a machine learning technique known in the art for training a model to, for example, identify or recognize patterns based at least in part on, in this case, health records corresponding to subjects know to be diagnosed with (or require a referral for) the disease, acute hepatic porphyria (AHP) as well as health records corresponding to subjects known not to have (or not to require a referral for) the disease, acute hepatic porphyria (AHP)). For purposes of training the model, such different health records may be “labeled” based on disease state or referral state. Supervised learning comprises training a model using such labeled training data.

In embodiments, supervised learning may be accomplished using scikit-learn (sklearn), a popular framework for training and evaluating machine learning models in Python. Further information is provided in: Pedregosa F, Varoquaux G, Gramfort A, et al. Scikit-learn: Machine Learning in Python. MACHINE LEARNING IN PYTHON: 6, the disclosure of which is incorporated herein in its entirety. For training sklearn-based classifiers, multiple linear and tree-based algorithms may be applied including, for example, Naive Bayes, KNeighbors, Logistic Regression, Decision Tree, Random Forest, Extra Tree, Support Vector Machines (SVMs), AdaBoost (Adaptive Boosting), XGBoost (extreme Gradient Boosting) and LightGBM (Light Gradient Boosting Machine). Other classifiers may be trained using automated machine learning (autoML), an end-to-end approach that streamlines many aspects of classifier development such as feature engineering, hyperparameter tuning, and ensembling.

In some embodiments, training and evaluating the model at step 145 involves training and testing machine learning models for referral and diagnosis using a held out test set and cross validation evaluations.

Some embodiments apply a round robin training technique to the model at step 145. By “round robin training technique,” it is meant that input data to the model (i.e., data used to train the model) is divided into multiple partitions such that certain partitions are used to train the model and the remaining partitions are used to generate predictions using the trained model. Such process may be iterated where partitions of data previously used to train the model are subsequently used to generate predictions using the trained model. Round robin training approaches may offer benefits including identifying which data sets used for training result in more accurate predictions.

In embodiments, evaluating the model at step 145 comprises assessing the accuracy of, or confidence in, predictions made by the model. That is, whether a prediction based on the model that the disease is present in (or a referral is warranted for) a subject represents a true positive or a false positive and/or whether a prediction based on the model that the disease is not present in (or a referral is not warranted for) a subject represents a true negative or a false negative, as well as how frequently such errors occur.

In embodiments, the model may be configured to generate information about the confidence of a prediction generated by the model. In such embodiments, training the model may comprise initially applying the model to obtain initial predictions, and using at least a subset of the initial predictions to further train the model, where the subset of initial predictions used to further train the model correspond to higher confidence predictions. That is, in some embodiments, training the model comprises generating a prediction as well as an indication of the degree of confidence that the prediction is accurate. After a model is trained to estimate the presence of, or recommend a referral for, the disease, acute hepatic porphyria (AHP), at step 145, the process moves to step 150. In embodiments, the referral model may comprise more than one models, for example, where each model corresponds to a medical center from which health records are obtained. In general, any number of referrals models may be generated or trained, and such number may vary, for example, vary based on the number of medical centers or other sources of health information used to train the model.

At step 150, the model is further trained and evaluated using the entire case and control patient cohort, i.e., the entire population of health records for identified at step 105 and selected at step 110. By “entire population,” it is meant health records corresponding to subjects diagnosed as having/not having or referred for/not referred for the disease, acute hepatic porphyria (AHP). In contrast, at step 145, an embodiment of the model is trained using only a subset of the available health records. At step 150, any convenient model generation method or model training method to train or refine the training of the model may be applied, including, for example, those described above in connection with step 145. Upon successful completion of step 150, a fully trained diagnostic model 155 is available, and first and second fully trained referral models 165 are available.

After model generation at step 150, the process can move to steps 165 and 170 to apply the first and second referral models generated at step 150. At step 170, the referral models may be run on, i.e., the referral models may be applied to a patient population, to predict whether a referral for evaluation of the disease, acute hepatic porphyria (AHP), is warranted for any patient in such population. In the embodiment depicted in FIG. 1, the referral model is applied to a patient population satisfying specific criteria. Any relevant criteria may be applied. In the case of step 170, for example, the patient population comprises patients that exhibit a specific symptom. Any relevant symptom may be applied. In the case of step 170, the patient population corresponds to patients exhibiting abdominal pain having occurred within the three years prior to the date on which the model is trained and applied, i.e., within the last three years. Exemplary first and second referral models 165, generated at step 150 and applied at step 170, are trained to output an estimate of whether a referral is warranted for a subject (i.e., based on a subject's health records) as well as an indication of the confidence in such estimate, i.e., a degree of confidence in a prediction. In the case of first and second referral models 165, information about the confidence of a prediction is indicated as a percentage. Upon applying the first and second referral model to the patient population of interest at step 170, the corresponding confidence score for each referral recommendation is evaluated at step 175. At step 175, it is determined whether: in the event that at step 170, application of the first and second referral models 165 indicates that a referral for a patient is warranted with a confidence score greater than 10%, such patent (i.e., health record) proceeds to step 160 for further processing via the diagnostic model.

While the embodiment depicted in FIG. 1 uses a 10% confidence score as a threshold at step 175, any confidence score or other criteria may be used as a threshold, and such may vary in different embodiments. Alternatively, at step 175, it is determined whether: in the event that at step 170, application of the first and second referral models 165 indicates that a referral for a patient is warranted with a confidence score less than 10%, such patent (i.e., health record) proceeds to step 185 where such patient (i.e., health record) is excluded from further processing on the grounds that the model estimates the patient does not have, or is very unlikely to be diagnosed with, the disease, acute hepatic porphyria (AHP). After a patient record is identified for further evaluation to estimate whether the disease is present (i.e., a patient record, for which the first and second referral models indicate a referral is warranted with a confidence score of greater than 10% at step 175), the process moves to step 160.

At step 160, the diagnosis model 155, generated/further trained at step 150, is applied to each health record that was not excluded by the referral model, as described above (i.e., the health records excluded via step 185). By applying the diagnosis model 155, it is meant that the diagnosis model 155 is used to estimate the presence of the disease, acute hepatic porphyria (AHP), in health records corresponding to patients who potentially have the disease. Like the first and second referral models 165, as described above, the exemplary diagnosis model 155, generated at step 150 and applied at step 160, is trained to output an estimate of the presence of a disease in a subject (i.e., based on a subject's health records) as well as an indication of the confidence in such estimate, i.e., a degree of confidence the model's estimate of the presence of the disease in the subject. In the case of diagnostic model 155, information about the confidence of a prediction is indicated as a percentage. After running the diagnostic model 155 on a health record at step 160, the process moves to step 180.

At step 180, the confidence score associated with an estimate regarding the presence of the disease in a subject is evaluated. At step 180, it is determined whether: in the event that at step 160, application of the diagnostic model 155 indicates that the patient is diagnosed with the disease with a confidence score greater than 50%, such patent (i.e., health record) proceeds to step 190 for further processing via clinical or biochemical confirmation. While the embodiment depicted in FIG. 1 uses a 50% confidence score as a threshold at step 180, any confidence score or other criteria may be used as a threshold, and such may vary in different embodiments. Alternatively, at step 180, it is determined whether: in the event that at step 160, application of the diagnostic model 155 indicates that the patient is diagnosed with the disease with a confidence score less than 50%, such patent (i.e., health record) proceeds to step 185 where such patient (i.e., health record) is excluded from further processing on the grounds that the model estimates the patient does not have, or is very unlikely to be diagnosed with, the disease, acute hepatic porphyria (AHP). After a patient record is identified for further evaluation to estimate whether the disease is present (i.e., a patient record, for which the diagnostic model indicates the patient is diagnosed with the disease with a confidence score of greater than 50% at step 180), the process moves to step 190.

At step 190, subjects corresponding to those health records, which are estimated by diagnosis model 155 to be diagnosed with the disease, acute hepatic porphyria (AHP), are subjected to confirmation of such diagnosis. That is, at step 190, further testing is applied to evaluate whether the diagnosis model 155 indicated a true positive or a false positive. Any applicable and/or relevant confirmation may be applied. For example, at step 190, the patient may undergo a biochemical test and/or a clinical examination in order to confirm the diagnosis of the disease, acute hepatic porphyria (AHP). In embodiments, biochemical tests and/or clinical examination to confirm a diagnosis of AHP may include, for example, hemin or urine lab test, e.g., to determine high dALA/PBG and clinical criteria versus normal dALA/PBG for negative AHP diagnosis. In the event the diagnosis is not confirmed, further evaluation of the subject may be conducted to confirm whether the diagnosis model 155 estimated a false positive. After confirmation at step 190 is performed, the process ends at step 195.

In the description of flow diagram 100 above, after model generation at step 150, the process applies and evaluates first and second referral models 165 at steps 170 and 175. However, in some cases, after model generation at step 150, embodiments of methods of the present invention may next apply and evaluate the results of the diagnosis model 155 without application of first and second referral models. That is, upon completion of step 150, embodiments of methods may move directly to steps 160 and 180, via diagnosis model 155.

In the description of flow diagram 100 above, at step 150, both first and second referral models 165 are generated for use in estimating whether referrals of subjects are warranted based on their health records. In alternative embodiments of methods according to the present invention, only a single referral model is generated, rather than two separate models.

FIG. 2 depicts a causal model diagram 200 that illustrates exemplary patient trajectories involving selection bias leading to a rare disease diagnosis, i.e., the patient journey from the first onset of AHP symptoms to an expert-assigned diagnosis. The embodiment of the present invention discussed in connection with FIG. 2 relates to diagnosing or referring a subject for the rare disease Acute Hepatic Porphyria (AHP), a heritable metabolic disorder of the heme biosynthesis pathway. Causal model diagram 200 relates to an exemplary embodiment of the present invention provided for illustrative purposes; the disease acute hepatic porphyria (AHP) is provided for illustrative purposes, and it is to be understood that the present invention is not limited to methods solely related to acute hepatic porphyria (AHP).

Referring to causal model 200, in general, the patient has the AHP risk gene 205, and once the symptoms are exhibited 210, e.g., disease complications are present 215, the patient is referred 220 to an expert clinician, such as a hepatology clinic, for example, for a thorough evaluation. Such a referral 220 is linked with number of selection biases including, for example, locality 230, insurance provider 235 and availability of medical expertise 240. Once the patient has undergone an expert evaluation 245 (based on referral 220), such as an evaluation at a hepatology clinic, the next step is a biochemical lab test to identify the Urinary and plasma aminolevulinic acid (ALA) and porphobilinogen (PBG) measurements, i.e., a lab order 250. If the patient complies with the lab orders 250, then finally an AHP diagnosis will be confirmed 255; i.e., diagnosis of whether the patient has, or does not have, AHP.

This causal model diagram 200 illustrates the complexity and biasing factors that culminate in the confirmation of a rare disease such as AHP. It highlights the importance of incorporating and/or addressing these factors in a predictive model, such as embodiments of the present invention configured to estimate the presence of a rare disease in a subject, i.e., a patient, to identify undiagnosed patients in a more timely way than any given health system might be able to diagnose these patients via the standard of care.

Two key stages along the patient journey as captured within a health system are seen in causal model 200. The first is recognition of possible AHP by a non-specialist and the corresponding decision to refer to a specialist 220. The second is testing and confirmation of a new diagnosis for AHP 255. Arriving at each of these two stages, referral to a specialist 220 and confirmation of a new diagnosis for AHP 255, are modeled separately in, respectively, a referral model(s) and a diagnosis model, in each case, described herein (e.g., as described in connection with steps 155 and 165 of FIG. 1). In embodiments, predictors for each model (i.e., disease specific features or indicators) may be slightly different. In some embodiments, a diagnosis model can only make AHP predictions on (i.e., be applied to) patients with some reasonable chance of being referred to an expert (i.e., patients for whom the referral model estimates some reasonably chance of a referral to a specialist being warranted), and may abstain from making predictions (i.e., being applied) to other patients.

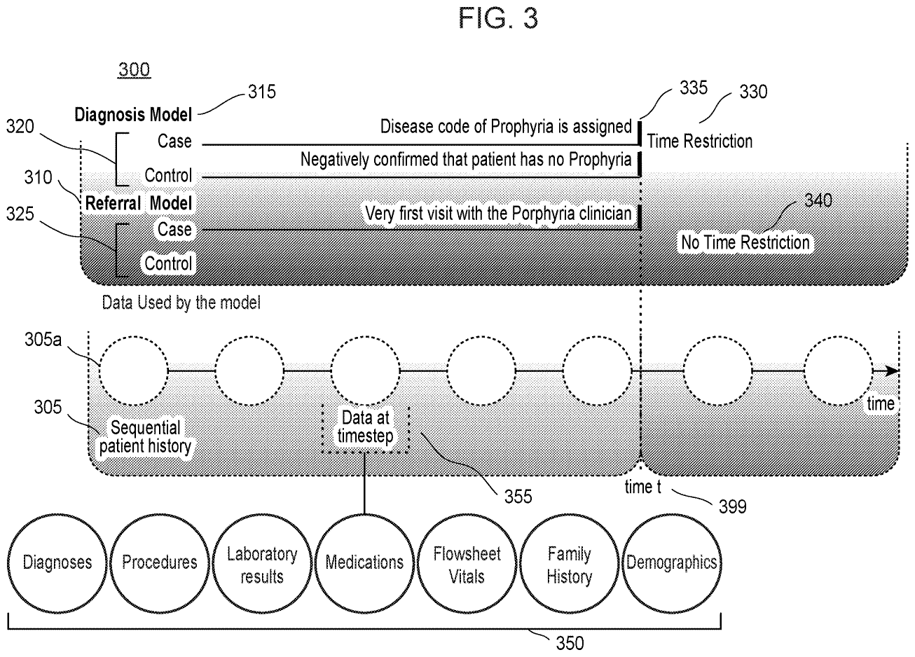

FIG. 3 depicts a flow diagram corresponding to an embodiment of a method of the present invention in the context of subject health records, i.e., sequential patient history of health events recorded in a health record. The embodiment of the present invention discussed in connection with FIG. 3 relates to diagnosing or referring a subject for the rare disease Acute Hepatic Porphyria (AHP), a heritable metabolic disorder of the heme biosynthesis pathway. Flow diagram 300 relates to an exemplary embodiment of the present invention provided for illustrative purposes; the disease acute hepatic porphyria (AHP) is provided for illustrative purposes, and it is to be understood that the present invention is not limited to methods solely related to acute hepatic porphyria (AHP).

In FIG. 3, flow diagram 300 illustrates the sequential patient history 305 with various clinically relevant time slicing, e.g., time slice 305a, on patient data available to referral model 310 and diagnosis model 315 (i.e., embodiments of those models described in connection with steps 155 and 165 of FIG. 1). In diagram 300, clinical events occurring in time 399 progress sequentially from the left hand side of the figure to the right hand side of FIG. 3. That is, FIG. 3 describes a sequential patient history with data at each timestamp collected from records of diagnosis, procedure, encounter, lab orders, medications, flow sheet vitals, demographics, and the like.

Embodiments of referral model 310 and diagnosis model 315 are developed using multicenter data 320, 325 (where such data may comprise a plurality of health records, such as sequential patient history 305) where two referral 310 and one diagnostic 315 machine learning based prediction models are generated. For diagnosis model 315, the conditions for confirmed AHP patient data time restriction 330 is based on the date (date 335) of disease code of Porphyria (i.e., AHP) is assigned and for the negative AHP patients the condition was the date (date 335) of negatively confirmed that patient has no Porphyria (AHP). For referral model 310 the condition for case patient data time restriction is the very first visit (date 335) with a Porphyria (AHP) clinician and there is no time restriction 340 for control data because from this patient pool the likelihoods are generated.

Flow diagram 300 depicts the sequential patient history 305 with data at each timestamp (e.g., data 355 at timestamp 305a) collected from records of diagnosis, procedure, encounter, lab orders/results, medications, flow sheet vitals, family history, demographics, etc. 350.

In addition to the aspects of the present disclosure described above, in some embodiments of the methods, the disease is acute hepatic porphyria. In some cases, the health records comprise structured electronic health records, unstructured electronic health records or combinations thereof. In embodiments, the health records originate from more than one medical center.

In other embodiments, the health information included within a health record comprises any relevant health information, such as one or more of: a diagnosis, information about a procedure, laboratory results, medication information, vitals information, family history or demographic information. In still other embodiments, modifying each health record of the plurality of health records to exclude certain health information comprises excluding health information subsequent to an occurrence of a clinically relevant event. In some cases, the occurrence of a clinically relevant event comprises one or more of: an assignment of a disease code or negative confirmation of the disease or a first visit to a specialist clinic.

In embodiments, potential indicators of the disease (also referred to as features or disease specific indicators or disease specific features) comprise any relevant medical observation, such as, for example, one or more of: tremor, hypertension, nausea, pancreatitis, dysuria, hallucinations, abdominal pain, sinus tachycardia, procedures on the stomach or increased sweating.

In other embodiments, training a machine learning model to estimate the presence of the disease based on the potential indicators of the disease using a first subset of the modified health records comprises utilizing one or more of stratified sampling, held out test set, cross validation, hyper parameter tuning or random grid search. In some cases, evaluating the accuracy of estimates of the presence of the disease comprises using any available technique for assessing the accuracy of a model, such as, for example, using one or more of held out test sets and cross validation evaluations. In other cases, further training the machine learning model using the plurality of health records comprises training the machine learning model using the plurality of health records in their entirety. In embodiments, clinically evaluating the presence of the disease comprises clinical evaluation of the subject by a specialist. In some cases, clinically evaluating the presence of the disease comprises conducting biochemical analysis. In certain cases, the biochemical analysis comprises one or more of identifying urinary and plasma amniolevulinic acid levels or identifying porphobilinogen (PBG) levels.

In certain embodiments, the publicly available databases comprise any available database or source of information regarding the applicable rare disease, such as, for example, one or more of a rare disease database or a semantic predictions database. As described in detail below, a semantic database may comprise the SemMedDB (NIH-NLM) database, and the rare disease database may comprise information obtained from one or more resources available via GARD (NCATS).

In embodiments, the subject belongs to an at-risk population for the disease. That is, the subject to be evaluated for diagnosis or whether a referral is warranted may be selected based on some predetermined criteria. Any applicable predetermined criteria may be applied, and such may vary. In some cases, the at-risk population comprises subjects exhibiting specified symptoms. For example, the specified symptoms comprise abdominal pain over a three-year period.

As described above, aspects of the present invention include training and applying a model to estimate whether a referral for a rare disease is warranted. In some cases, such machine learning model comprises a first referral model and a second referral model.

In embodiments, a machine learning model may comprise one or more of: a statistical model, a linear model, a computational model, a tree-based model, a convolutional neural network, an artificial neural network or a deep learning network, as such models and techniques for training and applying them are known in the art. In embodiments, a machine learning model may comprise linear and tree-based baseline models, autoML-based boosted and ensemble models, and deep learning models. In certain cases, training the machine learning model comprises one or more of: an unsupervised learning technique, a semi-supervised learning technique or a supervised learning technique.

As described above, the subject or subjects or patient or patients may be human. The subject or subjects may be male or female. The subject or subjects may be adult subjects or pediatric subjects or both, ranging, for example, from ten years old to 65 years old. The subject or subjects may have any body mass index and such may vary.

In embodiments, the disease may comprise one of: factor XIII deficiency, Hutchinson-Gilford progeria syndrome, Barth syndrome (BTHS), nephrogenic diabetes insipidus, congenital generalized lipodystrophy (also known as Berardinelli-Seip lipodystrophy), fibrolamellar hepatocellular carcinoma (FHCC), autoimmune polyendocrine syndrome type 1 (APS-1), cerebral creatine deficiency syndromes, cyclic neutropenia, goblet cell carcinoid, ablepharon-macrostomia syndrome (AMS), Alexander disease, Baller-Gerold syndrome (BGS), Bernard-Soulier syndrome (BSS), Caroli disease, Cleidocranial dysostosis (CCD), cold agglutinin disease (CAD), congenital afibrinogenemia, cutaneous T cell lymphoma (CTCL), cutis laxa, factor XI or plasma thromboplastin antecedent, factor XII deficiency, familial partial lipodystrophy, fatal insomnia, glycogen storage disease type VI (GSD VI), hypothalamic disease, keratosis follicularis spinulosa decalvans, Miller syndrome, opsoclonus myoclonus syndrome (OMS), paroxysmal nocturnal hemoglobinuria (PNH) or Crigler-Najjar syndrome. Techniques described herein may apply to other disease with similar rates of prevalence or incidence.

### Computer Implemented Embodiments

The various method and algorithm steps described in connection with the embodiments disclosed herein can be implemented as electronic hardware, computer software, or combinations of both. To clearly illustrate this interchangeability of hardware and software, various illustrative steps have been described above generally in terms of their functionality. Whether such functionality is implemented as hardware or software depends upon the particular application and design constraints imposed on the overall system applying a method according to the present disclosure. The described functionality can be implemented in varying ways for each particular application, but such implementation decisions should not be interpreted as causing a departure from the scope of the disclosure.

The various illustrative steps, components, and computing systems (such as devices, databases, interfaces, and engines) described in connection with the embodiments disclosed herein can be implemented or performed by a machine, such as a general purpose processor, a graphics processor unit, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor can also include primarily analog components. A computing environment can include any type of computer system, including, but not limited to, a computer system based on a microprocessor, a graphics processor unit, a mainframe computer, a digital signal processor, a portable computing device, a personal organizer, a device controller, and a computational engine within an appliance, to name a few.

The steps of a method, process, or algorithm described in connection with the embodiments disclosed herein can be embodied directly in hardware, in a software module executed by a processor, or in a combination of the two. A software module, engine, and associated databases can reside in memory resources such as in RAM memory, FRAM memory, flash memory, ROM memory, EPROM memory, EEPROM memory, registers, hard disk, a removable disk, a CD-ROM, or any other form of non-transitory computer-readable storage medium, media, or physical computer storage known in the art. An external storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

As described in detail above, embodiments of the present invention relate to computer-implemented methods for improving the diagnosis of rare diseases using electronic health records. Aspects of the present invention include methods of training a model to estimate the presence of a disease in a subject comprising: obtaining a plurality of health records comprising health information about individuals, modifying each health record of the plurality of health records to exclude certain health information, identifying potential indicators of a disease based on publicly available databases, where the disease is a rare disease (e.g., a disease that occurs in approximately one in 100,000 individuals), and the potential indicators comprise health information present in the health records, training a machine learning model to estimate the presence of the disease based on the potential indicators of the disease using a first subset of the modified health records, evaluating the accuracy of estimates of the presence of the disease based on the potential indicators of the disease made by the machine learning model using a second subset of the modified health records, and further training the machine learning model using the plurality of health records and based at least in part on the results of evaluating the accuracy of the machine learning model. Aspects of the present invention further include methods of inferring the presence of a disease in one or more subjects; methods of clinically evaluating the presence of a disease in one or more subjects; methods of training a model to recommend a referral for evaluating the presence of a disease in one or more subjects; as well as methods of recommending a referral for evaluating the presence of a disease in one or more subjects.

### Systems for Improving the Diagnosis of Rare Diseases Using Electronic Health Records Data

As summarized above, aspects of the present disclosure include systems for improving the diagnosis of rare diseases using electronic health records data. Systems according to certain embodiments comprise a processor comprising memory operably coupled to the processor, wherein the memory comprises instructions stored thereon, which, when executed by the processor, cause the processor to execute steps corresponding to the subject methods described herein.

In some embodiments of systems according to the present disclosure comprise: a processor comprising memory operably coupled to the processor, wherein the memory comprises instructions stored thereon, which, when executed by the processor, cause the processor to: obtain a plurality of health records comprising health information about individuals; modify each health record of the plurality of health records to exclude certain health information; identify potential indicators of a disease based on publicly available databases, wherein the disease is a disease that occurs in approximately one in 100,000 individuals, and the potential indicators comprise health information present in the health records; train a machine learning model to estimate the presence of the disease based on the potential indicators of the disease using a first subset of the modified health records; evaluate the accuracy of estimates of the presence of the disease based on the potential indicators of the disease made by the machine learning model using a second subset of the modified health records; and further train the machine learning model using the plurality of health records and based at least in part on the results of evaluating the accuracy of the machine learning model.

In other embodiments of systems according to the present disclosure, the memory further comprises instructions stored thereon, which, when executed by the processor, cause the processor to: obtain a health record comprising health information about a subject; apply the trained model to the health record of the subject to estimate the presence in a disease in the subject; and estimate the presence of the disease in the subject based on the results of applying the model. In other embodiments of systems according to the present disclosure, the memory further comprises instructions stored thereon, which, when executed by the processor, cause the processor to: estimate the presence of a disease in the subject, wherein the estimate of the presence of the disease comprises a confidence score; and indicate a determination to clinically evaluate the presence of the disease in the subject, in the event the confidence score exceeds a predetermined threshold. In other embodiments of systems according to the present disclosure, the memory further comprises instructions stored thereon, which, when executed by the processor, cause the processor to: obtain health records comprising health information about a plurality of subjects; apply the trained model to the health records to estimate the presence of a disease in each subject of the plurality of subjects; and infer the presence of the disease in each subject of the plurality of subjects based on the results of applying the model. In other embodiments of systems according to the present disclosure, the memory further comprises instructions stored thereon, which, when executed by the processor, cause the processor to: estimate the presence of a disease in a plurality of subjects, wherein each estimate of the presence of the disease in each subject of the plurality of subjects comprises a confidence score; and indicate a determination to clinically evaluate the presence of the disease in each subject, in the event the confidence score for such subject exceeds a predetermined threshold.

In some embodiments of systems according to the present disclosure comprise: a processor comprising memory operably coupled to the processor, wherein the memory comprises instructions stored thereon, which, when executed by the processor, cause the processor to: obtain a plurality of health records comprising health information about individuals; modify each health record of the plurality of health records to exclude certain health information; identify potential indicators of a disease based on publicly available databases, wherein the disease is a disease that occurs in approximately one in 100,000 individuals, and the potential indicators comprise health information present in the health records; train a machine learning model to recommend a referral for evaluating the presence of a disease based on the potential indicators of the disease using a first subset of the modified health records; evaluate the accuracy of the machine learning model based on the potential indicators of the disease using a second subset of the modified health records; and further train the machine learning model using the plurality of health records and based at least in part on the results of evaluating the accuracy of the machine learning model. In other embodiments of systems according to the present disclosure, the memory further comprises instructions stored thereon, which, when executed by the processor, cause the processor to: obtain a health record comprising health information about a subject; apply the trained model to the health record of the subject to recommend a referral for evaluating the presence of a disease in the subject; and recommend a referral for evaluating the presence of a disease in the subject based on the results of applying the model. In other embodiments of systems according to the present disclosure, the memory further comprises instructions stored thereon, which, when executed by the processor, cause the processor to: determine whether a referral for evaluating the presence of a disease in a subject is recommended, wherein the recommendation for a referral comprises a confidence score and wherein the model comprises a referral model; obtain a plurality of health records comprising health information about individuals; modify each health record of the plurality of health records to exclude certain health information; identify potential indicators of a disease based on publicly available databases, wherein the disease is a disease that occurs in approximately one in 100,000 individuals, and the potential indicators comprise health information present in the health records; train a machine learning diagnosis model to estimate the presence of the disease based on the potential indicators of the disease using a first subset of the modified health records; evaluate the accuracy of estimates of the presence of the disease based on the potential indicators of the disease made by the machine learning diagnosis model using a second subset of the modified health records; further train the machine learning diagnosis model using the plurality of health records and based at least in part on the results of evaluating the accuracy of the machine learning model; apply the trained diagnosis model to a health record of the subject to estimate the presence in a disease in the subject, in the event the confidence score generated by the referral model exceeds a predetermined threshold; and estimate the presence of the disease in the subject based on the results of applying the diagnosis model. In other embodiments of systems according to the present disclosure, the memory further comprises instructions stored thereon, which, when executed by the processor, cause the processor to: estimate the presence of a disease in a subject using the diagnosis model, wherein the estimate of the presence of the disease comprises a confidence score; and indicate a determination to clinically evaluate the presence of the disease, in the event the confidence score exceeds a predetermined threshold. In other embodiments of systems according to the present disclosure, the memory further comprises instructions stored thereon, which, when executed by the processor, cause the processor to: obtain health records comprising health information about a plurality of subjects; apply the trained model to the health records to recommend a referral for evaluating the presence of a disease in each subject of the plurality of subjects; and recommend a referral for evaluating the presence of a disease in each subject of the plurality of subjects based on the results of applying the model. In other embodiments of systems according to the present disclosure, the memory further comprises instructions stored thereon, which, when executed by the processor, cause the processor to: determine whether a referral for evaluating the presence of a disease in each subject of a plurality of subjects is recommended, wherein the recommendation for a referral comprises a confidence score and wherein the model is a referral model; obtain a plurality of health records comprising health information about individuals; modify each health record of the plurality of health records to exclude certain health information; identify potential indicators of a disease based on publicly available databases, wherein the disease is a disease that occurs in approximately one in 100,000 individuals, and the potential indicators comprise health information present in the health records; train a machine learning diagnosis model to estimate the presence of the disease based on the potential indicators of the disease using a first subset of the modified health records; evaluate the accuracy of estimates of the presence of the disease based on the potential indicators of the disease made by the machine learning diagnosis model using a second subset of the modified health records; further train the machine learning diagnosis model using the plurality of health records and based at least in part on the results of evaluating the accuracy of the machine learning model; apply the diagnosis model to health records of each subject of the plurality of subjects to estimate the presence of a disease of each subject of the plurality of subjects, in the event the confidence score generated by the referral model exceeds a predetermined threshold; and estimate the presence of the disease in each subject of the plurality of subjects based on the results of applying the model. In other embodiments of systems according to the present disclosure, the memory further comprises instructions stored thereon, which, when executed by the processor, cause the processor to: estimate the presence of a disease in a plurality of subjects, wherein each estimate of the presence of the disease comprises a confidence score; and indicate a determination to clinically evaluate the presence of the disease for each subject of the plurality of subjects, in the event the confidence score exceeds a predetermined threshold.

Some embodiments may further comprise a display device, e.g., for displaying results of estimating, referring, diagnosing or the like. Any convenient display device, such as a liquid crystal display (LCD), light-emitting diode (LED) display, plasma (PDP) display, quantum dot (QLED) display or cathode ray tube display device. The processor and/or memory may be operably connected to the display device, for example, via a wired, such as a Universal Serial Bus (USB) connection, or wireless connection, such as a Bluetooth connection.

Aspects of the present disclosure further include non-transitory computer readable storage media for improving the diagnosis of rare diseases using electronic health records data. non-transitory computer readable storage media according to certain embodiments comprise one or more algorithms corresponding to the subject methods described herein.

### Utility

The subject methods and systems find use in a variety of applications where it is desirable to diagnose disease, in a more timely and accurate manner than presently available. In some embodiments, the methods and systems described herein find use in clinical settings such as any clinical setting where traditional, clinical or biochemical testing according to prevailing standards of care may be applied. In other embodiments, the methods and systems described herein find use in remote medicine settings, where specialized medical services capable of providing accurate diagnoses for one or more rare diseases may be newly enabled by application of the present methods and systems. In addition, the subject methods and systems find use in improving the effectiveness and timeliness of diagnosing rare disease.

In some cases, the subject methods and systems may find use in the context of offsetting the financial and quality of life-related costs resulting from the burden of undiagnosed disease. For example, healthcare payors (public and private insurers) may benefit from supporting timely diagnosis and the avoidance of unnecessary tests, procedures and their complications; electronic health record vendors may benefit from embedding a clinical decision support tool in product offerings; biopharmaceutical companies investigating rare diseases may benefit from, for example, utilizing the methods and systems herein to optimize clinical trial recruitment for regulatory demonstrations of efficacy and/or safety; health systems may benefit from applying the present invention to improve healthcare resource utilization and allocation; and disease advocacy groups may benefit from improved ability to identify patients affected by rare disease.

The following is offered by way of illustration and not by way of limitation.

### Experimental

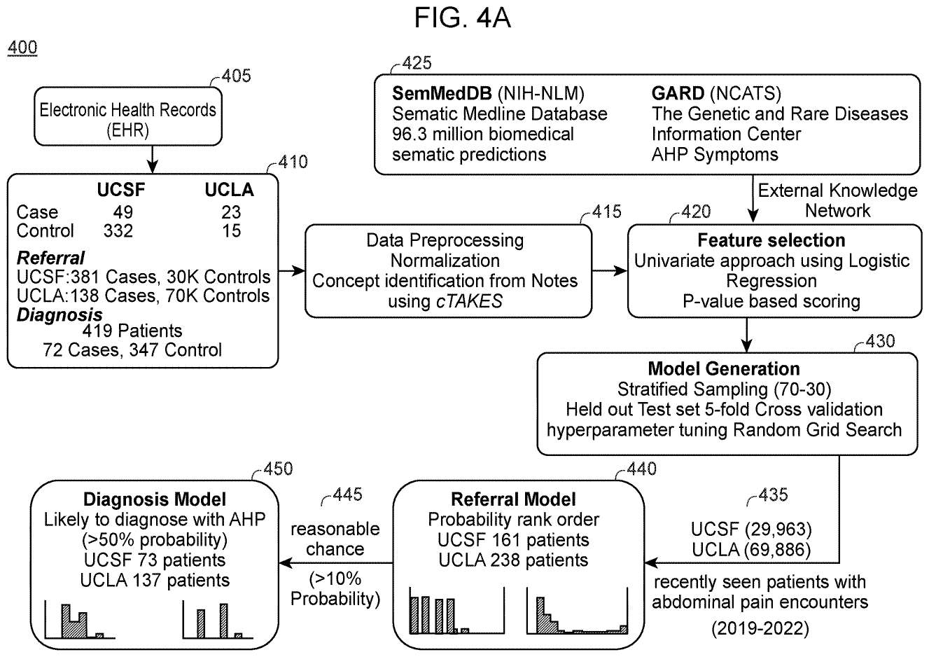

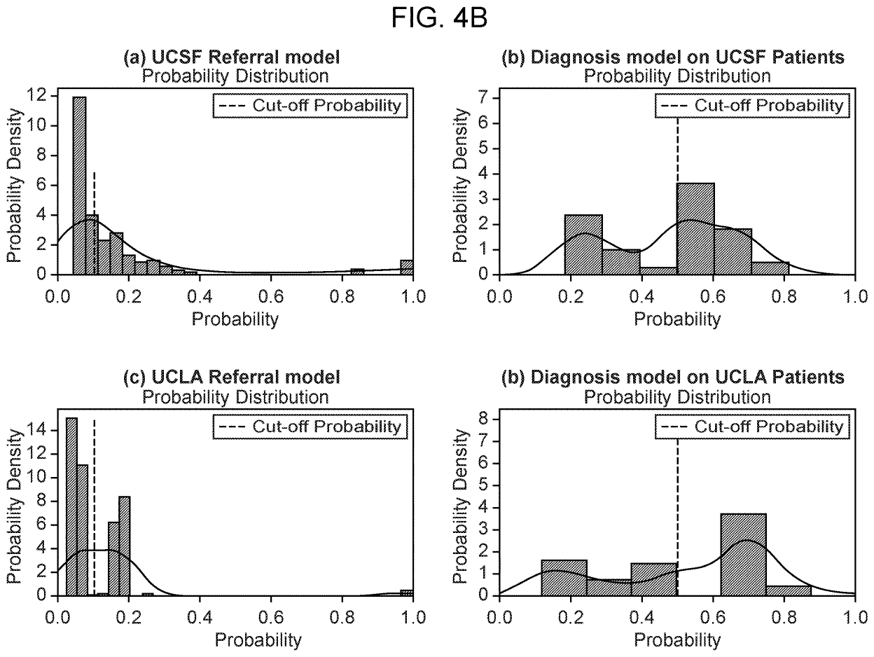

FIG. 4A illustrates application of an embodiment of a method according to the present invention for estimating the presence of the disease AHP in a subject and estimating whether a referral to a specialist for evaluation is warranted. The embodiment of the present invention depicted in FIG. 4A relates to diagnosing or referring a subject for the rare disease Acute Hepatic Porphyria (AHP), a heritable metabolic disorder of the heme biosynthesis pathway. The embodiment depicted in FIG. 4A is an exemplary embodiment of the present invention provided for illustrative purposes; the disease acute hepatic porphyria (AHP) is also provided for illustrative purposes, and it is to be understood that the present invention is not limited to methods solely related to acute hepatic porphyria (AHP). The embodiment of the present invention depicted in FIG. 4A presents an application in connection with a retrospective cross-sectional study of patients seen at two large, urban, tertiary care academic medical centers in the United States, University of California, Los Angeles and University of California, San Francisco.

As discussed in detail below, FIG. 4A presents results of applications of embodiments of the present invention in the following context. Two study cohorts, namely referral and diagnosis, were created for generating the prediction models. At UCSF, electronic health record (EHR) data from 2012-2022 was used in which for the referral model AHP cases were clinically confirmed by experts and controls were patients within the age range of 10-65 and at least one abdominal pain encounter. For the diagnosis model, AHP cases were determined by high dALA/PBG and clinical criteria, controls were patients with normal dALA/PBG. At UCLA, EHR data from 2019-2022 was used in which for referral model AHP cases where patients with at least one ICD 9/10 code of AHP or use of hemin or urine lab test and controls were the same as UCSF. For the diagnosis model, AHP cases were determined by high dALA/PBG and clinical criteria, controls were patients with normal dALA/PBG or no genetic mutation. At inference time, the referral control cohort is used to identify new patients with referral and diagnostic probability.

Two referral and one diagnosis models were developed and their performance was evaluated using Accuracy, F-Score, sensitivity, specificity, and new patients were identified from the undiagnosed population.

The UCSF referral cohort represented 381 cases and 29,963 control patients. The UCLA referral cohort represented 366 cases and 69,886 control patients. The diagnosis cohort from the two centers together represented 419patients with 72 cases (49-UCSF, 23-UCLA) and 347 control (332-UCSF, 15-UCLA) patients. The referral models were performed with Accuracy [89-93%] and F-score [86-91%]. The diagnosis model was performed with Accuracy and F-score [92.07%]. Using the referral model 168 patients from UCSF and 283 patients from UCLA were identified with more than 10% probability. Using these patients, the diagnosis model identified 98 patients from UCSF and 124 patients from UCLA with more than 50% probability.

In this multi-center case-control study machine learning prediction models identified AHP patients from a large pool of potential patients. These results suggest that carefully curated EHR data modeled using machine learning can predict the very rare occurrence of diseases in patients.

Next, the individual steps seen in FIG. 4A are discussed.

In connection with the embodiment depicted in FIG. 4A, two referral machine learning-based prediction models (one for each of two medical centers, UCSF and UCLA) and one diagnostic machine learning-based prediction model are generated. The medical center-specific referral models are used to find the likelihood of patients having the rare disease and to be biochemically tested for confirmation. The diagnosis model is used to evaluate the likelihood of a positive confirmation from the biochemical test. Since the rare disease is challenging to early diagnose a 10 percent probability cut-off for the referral model was applied and a 50 percent probability cut-off for the diagnosis model was applied.

In FIG. 4A, flow diagram 400 illustrates an exemplary machine learning models generation pipeline. The flow diagram starts at step 405, where patient data from two medical centers, UCSF and UCLA, is obtained. Patient data comprises health data corresponding to patients. In the embodiment depicted in FIG. 4A, such health data comprises electronic health records (EHR). In the embodiment depicted in FIG. 4A, such electronic health records (EHR) date from 2012-22 for UCSF data and 2019-22 for UCLA data, and patients ranged in age from ten to 65. Step 405 of flow diagram 400 is analogous to step 105 of flow diagram 100 seen in FIG. 1. After completing step 405, the process moves to step 410.

At step 410, two datasets were created, each designed to train a different prediction algorithm, referral algorithms and a diagnosis algorithm. The first dataset is a referral dataset that comprises data related to: among all patients who could have been referred for evaluation by an AHP clinical specialist, who was referred? The second dataset is a diagnosis dataset that comprises data related to: among all patients who were referred and seen by an AHP specialist, who was confirmed as having AHP?

In connection with generating a diagnosis model (i.e., a model trained to estimate the presence of the disease AHP in a subject based on the subject's health records), 419 patients (72 cases and 347 controls) were identified for use at step 410. In the context of the diagnosis model, a “case” refers to a health record for which the corresponding subject received a diagnosis for AHP, and a “control” refers to a health record for which the corresponding subject did not receive a diagnosis for AHP (i.e., received affirmative confirmation that the subject did not have AHP).

In connection with generating a referral model (i.e., a model trained to estimate whether a referral to a specialist to evaluate the presence of the disease AHP in a subject based on the subject's health records), 381 cases and approximately 30,000 controls were identified for use at step 410 from health records originating from UCSF; and 366 cases and approximately 70,000 controls were identified for use at step 410 from health records originating from UCLA. In the context of the referral model, a “case” refers to a health record for which the corresponding subject received a referral to a specialist in connection with evaluation of AHP, and a “control” refers to a health record for which the corresponding subject did not receive a referral to a specialist in connection with evaluation of AHP.

The UCSF referral cohort represented 381 cases and 29,963 control patients. The UCLA referral cohort represented 366 cases and 69,886 control patients. The diagnosis cohort from the two centers together represented 419patients with 72 cases (49-UCSF, 23-UCLA) and 347 control (332-UCSF, 15-UCLA) patients. The diagnosis cohorts with case and control together were used as the cases of each referral model. For the diagnosis cohort, the UCSF case consisted of 77.55% of female and 22.45% of male patients with 38.70 ±18.69 as the median age. The UCSF referral cohort case had an average body mass index of 23.92±1.23 and represented 67.34% White individuals, 0.81% Asian individuals, 0.20% Black individuals and 31.65% from other races. The UCSF control consisted of 45.78% of females and 54.22% of male patients with 40.14±18.63 as the median age. The UCSF control had an average body mass index of 29.16±17.98 and represented 52.71% White individuals, 0.18% Asian individuals, 0.06% Black individuals and 47.05% from other races. The UCLA case consisted of 86.95% of female and 13.05% of male patients with 40±15.31 as the median age. The UCLA case had an average body mass index of 27.47 ±6.32 and represented 52.17% White individuals, 0.086% Black individuals and 47.74% from other races.

The UCLA control consisted of 80.00% of females and 20.00% of male patients with 43+17.62 as the median age. The UCLA control had an average body mass index of 28.10±7.30 and represented 80.00% White individuals, 0.06% Black individuals and 19.94% from other races. Among the confirmed AHP cases from UCSF, 53.06% of patients had PBG and ALA lab test results, 36.73% of patients had Total porphyrins or Porphyrins fractionated, 12.24% had panhematin infusions, 38.77% had acute recurrent attacks of abdominal pain, 28.57% had family history records and 08.16% had psychiatry diagnosis code assigned. Among the confirmed AHP cases from UCLA, 78.26% of patients had PBG and 69.56% had ALA lab test results, 56.52% of patients had Total porphyrins or Porphyrins fractionated, 30.43% had panhematin infusions, 82.60% had acute recurrent attacks of abdominal pain, 13.04% had family history records and 26.08% had psychiatry diagnosis code assigned.

After step 410, the process moves to step 415.

At step 415, preprocessing, normalization, concept identification and frequency based feature (i.e., indicator) value generation are each applied with respect to the health records identified at step 410. Health records may comprise standard structured electronic health records (EHR) data (demographics, encounters, medications, labs, procedures). All clinical concepts present in clinical notes were extracted using automated tools. Specifically, in the embodiment depicted in FIG. 4A, concept identification is conducted using cTAKES As-A-Services tool (Version 3.1.1), a publicly available software package that stands for “clinical Text Analysis Knowledge Extraction System” and provides a natural language processing system for extraction of information from electronic medical record clinical free-text. cTAKES is available online at: https://ctakes.apache.org/index.html. Further details regarding cTAKES are provided in: Savova, Guergana; Masanz, James; Ogren, Philip; Zheng, Jiaping; Sohn, Sunghwan; Kipper-Schuler, Karin and Chute, Christopher. 2010. Mayo Clinic Clinical Text Analysis and Knowledge Extraction System (cTAKES): architecture, component evaluation and applications. JAMIA 2010; 17:507-513 doi:10.1136/jamia.2009.001560, the disclosure of which is incorporated herein in its entirety. In the embodiment depicted in FIG. 4A, cTAKES is deployed for processing and frequency-based feature value generation. While cTAKES software is used at step 415 in the embodiment depicted in FIG. 4A, any other convenient software package capable of data preprocessing and/or normalization and/or concept identification may be employed in other embodiments as needed. After step 415, the process moves to step 420.

At step 420, feature selection is performed to identify potential disease specific features present in the health records obtained at step 405. In embodiments, any convenient feature selection technique or techniques may be applied to identify health record features that may be predictive of disease diagnosis or referral. In the embodiment depicted in FIG. 4A, at step 420, feature selection is accomplished by applying univariate feature selection using logistic regression, and a p-value based cut-off is used to reduce the feature space. Selected features for modeling had to meet at least one of the following criteria: statistical significance in an unadjusted logistic regression model; selection by a clinical domain expert; or association with AHP based on scientific knowledge extracted from publicly available data (i.e., SemMedDB or GARD, as described in detail below).

In addition, in the embodiment depicted in FIG. 4A, feature selection is further augmented by incorporating information from an external knowledge network via step 425. That is, external biomedical and clinical knowledge is incorporated into the feature selection process at step 425. At step 425, relevant information is accessed from publicly available databases regarding characteristics of the rare disease, AHP. For feature selection, external biomedical and clinical knowledge regarding AHP is incorporated from SemMedDB, a semantic MEDLINE database listing PubMed scale biomedical semantic predications, and the Genetic and Rare Diseases (GARD) data of AHP. That is, at step 425, the publicly available SemMedDB (NIH-NLM) database is accessed for semantic relations information relevant to identifying disease specific features present in health records. SemMedDB is the Semantic Medline Database with 96.3 million biomedical semantic predictions. Further details regarding the SemMedDB database are provided in: Kilicoglu H, Shin D, Fiszman M, Rosemblat G, Rindflesch TC. SemMedDB: a PubMed-scale repository of biomedical semantic predications. Bioinformatics. 2012 Dec. 1;28(23):3158-60. doi: 10.1093/bioinformatics/bts591. Epub 2012 Oct. 8. PMID: 23044550; PMCID: PMC3509487, the disclosure of which is incorporated herein in its entirety. Also at step 425, the publicly available GARD (NCATS) information center is accessed for AHP symptom and condition information relevant to identifying disease specific features present in health records. GARD refers to Genetic and Rare Diseases and the GARD Information center is public health resource that provides information for patients regarding rare diseases. Information obtained from SemMedDB and GARD at step 425 is incorporated, using any convenient technique, into selection of disease specific features at step 420. After disease specific features are selected at step 420, the process moves to step 430.

At step 430, one or more models are generated for use estimating the presence of the disease in a patient; i.e., a diagnosis model is generated. Also at step 430, one or more models are generated for use estimating whether a referral to a specialist is warranted for a patient; i.e., a referral model is generated. Specifically, at step 430, the features selected at step 420 are identified in the health records obtained at step 405 and the patient data was fitted for one or more models using any convenient model generation or model training approach. Both classic machine learning and deep learning models may be trained to predict which patients will be referred (referral model) and which patients will be diagnosed (diagnosis model) among those who are referred.

At step 430, the machine learning algorithms AdaBoost and Logistic Regression were used for model building. Machine learning models were generated and performance was computed using the scikit-learn Python library (scikit-learn 1.0.2) in Python (version 3.7). Further details regarding this aspect are provided in: 12. Pedregosa F, Varoquaux G, Gramfort A, et al. Scikit-learn: Machine Learning in Python. MACHINE LEARNING IN PYTHON: 6, the disclosure of which is incorporate herein in its entirety.

Model generation approaches of interest, included, for example, stratified sampling, held out test set (e.g., with any convenient division between training and testing sets, such as, for example, a 70% training set and 30% testing set), cross validation (5-fold), hyper parameter tuning and random grid search. Model generation is performed based on the features selected at step 420 using all available health records obtained at step 405 and identified at step 410; the referral case and control health records being used to generate the referral model, and the diagnosis case and control health records being used to generate the diagnosis model.

On an initial run of the diagnosis algorithm, six AHP patients were identified with a markedly elevated urine ALA/PBG, previously unknown to the UCSF team maintaining an AHP registry. Such patients' records were added to the training dataset and the models were re-trained.

In connection with training the diagnosis and referral models, supervised machine learning (ML) was used to train all classifiers using scikit-learn (sklearn), a popular framework for training and evaluating machine learning models in Python. For sklearn-based classifiers, multiple linear and tree-based algorithms were trained including Naive Bayes, KNeighbors, Logistic Regression, Decision Tree, Random Forest, Extra Tree, Support Vector Machines (SVMs), AdaBoost (Adaptive Boosting), XGBoost (eXtreme Gradient Boosting) and LightGBM (Light Gradient Boosting Machine). A second set of classifiers were trained using automated machine learning (autoML), an end-to-end approach that streamlines many aspects of classifier development such as feature engineering, hyperparameter tuning, and ensembling. In addition, using n-gram features, 75 different classifiers were trained, including Random Forest and XGBoost. The open-source package autoGluon9to was used to train these classifiers. For the autoML classifiers, AutoGluon version 0.3.1 was used. The generated models include baseline classifiers as well as algorithm ensembles using stacking and bagging. In connection with the embodiment depicted in FIG. 4A, these classifiers were developed on the 11th generation Intel Core i5-1135G7 @ 2.40 GHz 1.38 GHz 64-bit processor with 8 GB RAM.

After the diagnosis model and referral model are generated, i.e., trained, at step 430, the process moves to step 440, via step 435.

During inference time (i.e., applying the two referral models and the diagnosis model), as described in detail below, the referral models were applied to the patient population satisfying specific criteria (symptom: abdominal pain, occurrence: last 3 years) and identifying a patient pool with at least ten percent likelihood flowingly run diagnosis model on the referral model patient cohort and identify patient pool with at least 50 percent likelihood for having the disease.

At step 440, the referral model generated at step 430 is applied to a subset of the health records obtained at step 405. As noted in transition 435, at step 440, the referral model is applied to those health records of the control health records for referrals (i.e., the 30,000 UCSF control records and 70,000 UCLA control records noted at step 410), for which the patient was recently seen (i.e., within the last three years) for abdominal pain. That is, at step 440, the referral model is applied to those health records, of the referral control health records identified at step 410, that indicate an abdominal pain encounter within the last three years. While the embodiment shown used a recent encounter for abdominal pain as criteria for selecting which health records to apply the model to, in general, any specific criteria could be applied. Application of the referral model at step 440 results in a health records being ordered based on likelihood or probability that a referral is warranted for each patient associated with a health record. Application of the referral model at step 440 estimated that a referral to a specialist for the rare disease AHP would be warranted for 161 health records from the UCSF population and 238 health records from the UCLA population.

After the referral model is applied to selected health records at step 440, the process moves to step 450, via transition 445.

At step 450, the diagnosis model is applied to a subset of the health records identified at step 440. Transition 445 indicates that the diagnosis model is applied to only those health records for which there is a reasonable chance that the patient associated with the health record would be diagnosed with the rare disease AHP. In the embodiment depicted in FIG. 4A, the diagnosis model is applied to those health records for which the referral model indicated a greater than 10% likelihood or probability of that a referral to a specialist for AHP is warranted for the subject associated with the health record. While the embodiment shown used a threshold of 10% likelihood, in general any threshold or other criteria could be applied for selecting which health records the diagnosis model should be applied to.

At step 450, application of the diagnosis model to health records results in an estimate of the probability that the patient associated with a health record is likely to be diagnosed with the rare disease AHP. At step 440, those patients with a greater than 50% likelihood of diagnosis with AHP are flagged as likely having AHP. While the embodiment shown used a threshold of 50% likelihood, in general any threshold or other criteria could be applied for selecting which health records are associated with a likely AHP diagnosis. Application of the referral model at step 440 resulted in an estimate that 73 health records from the UCSF population and 137 health records from the UCLA population are associated with patients that would have a positive diagnosis for AHP.

Model performance (i.e., performance of the diagnosis model as well as the referral models) was evaluated using the following performance metrics: sensitivity, specificity, accuracy and f-score.

The UCSF referral model Ada Boost performed best with Accuracy and F-score [91.02%] with 92.18% sensitivity, 84.63% specificity, and 83.91% positive predictive value (PPV). The UCLA referral model Logistic Regression performed best with Accuracy [89.41%] and F-score [86.12%] with 91.08% sensitivity, 79.12% specificity, and 86.19% positive predictive value (PPV). For diagnosis model Logistic Regression performed best with Accuracy and F-score [92.07%] with 95.60% sensitivity, 89.72% specificity and 92.05% positive predictive value (PPV).

For model inference, clinical criteria of Age [10-65] with at least one occurrence of Abdominal Pain in the last 3 years from [2019-2022] were used.

For UCSF the inference patient cohort consisted of 29,963 patients and for UCLA the cohort size was 69,880 patients. Using the referral model 168 patients from UCSF and 283 patients from UCLA were identified with more than 10% model prediction probability. Using these patients, the diagnosis model was evaluated and 98 patients from UCSF and 124 patients from UCLA were identified with more than 50% probability.

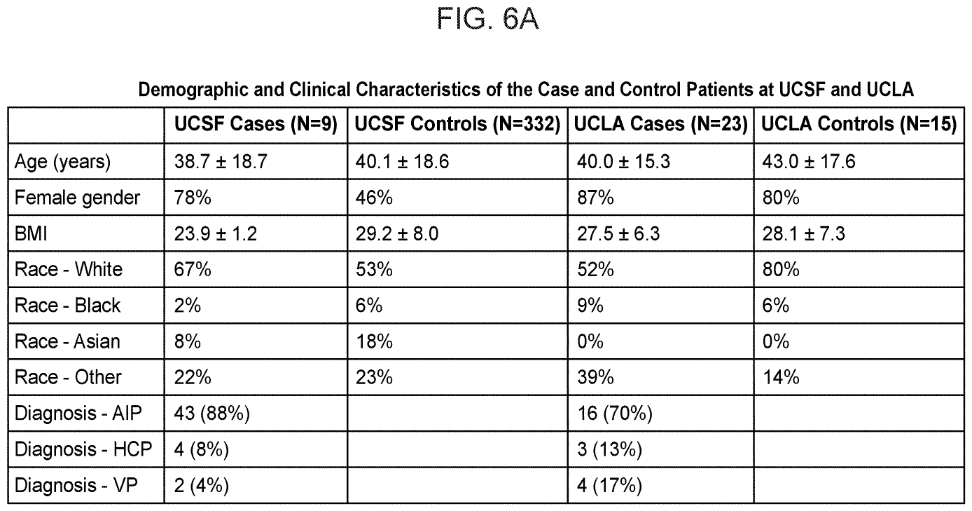

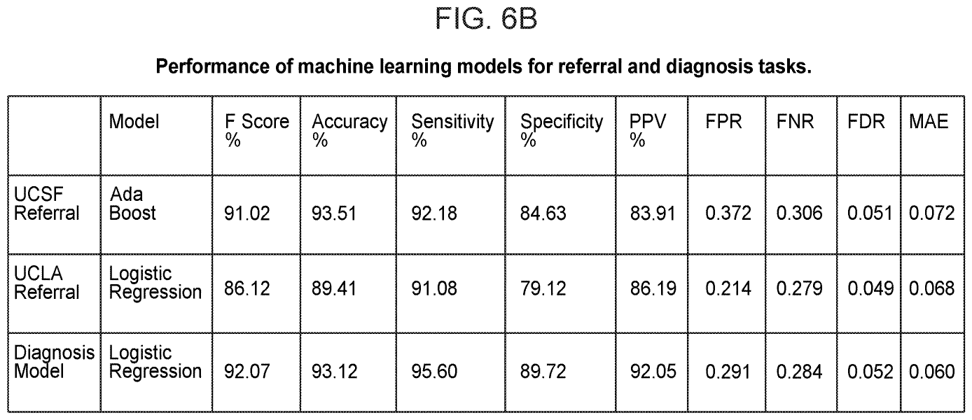

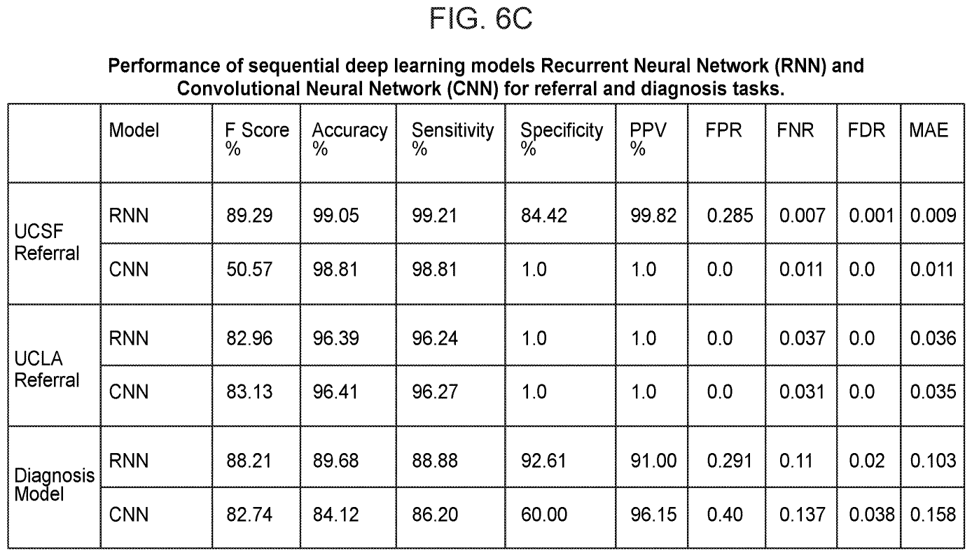

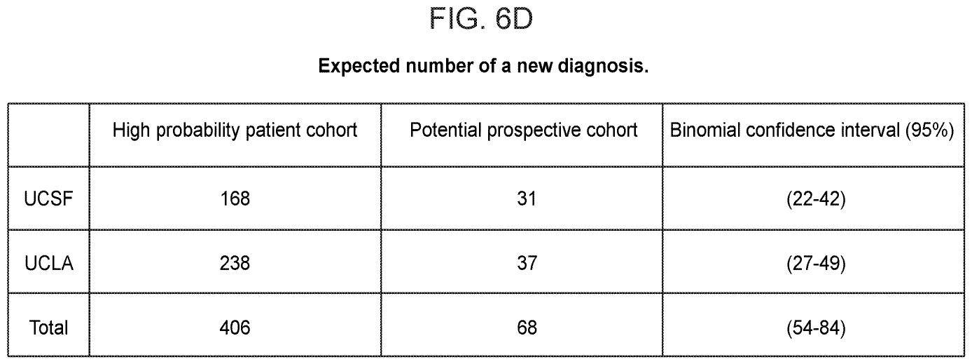

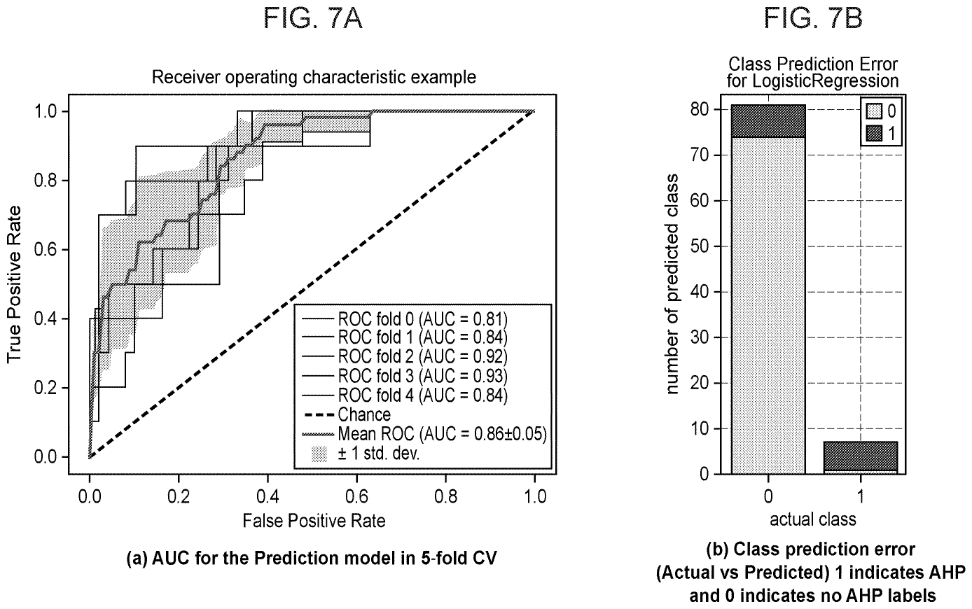

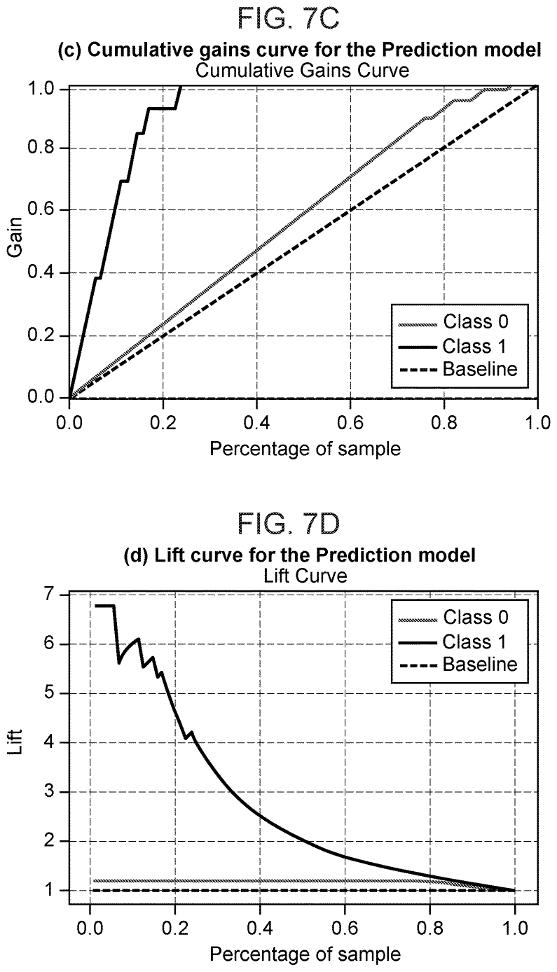

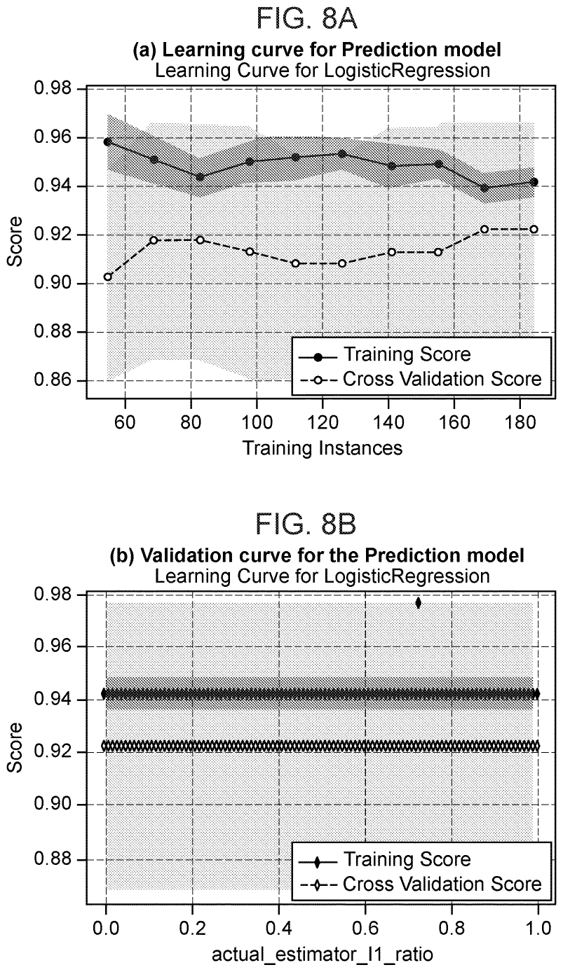

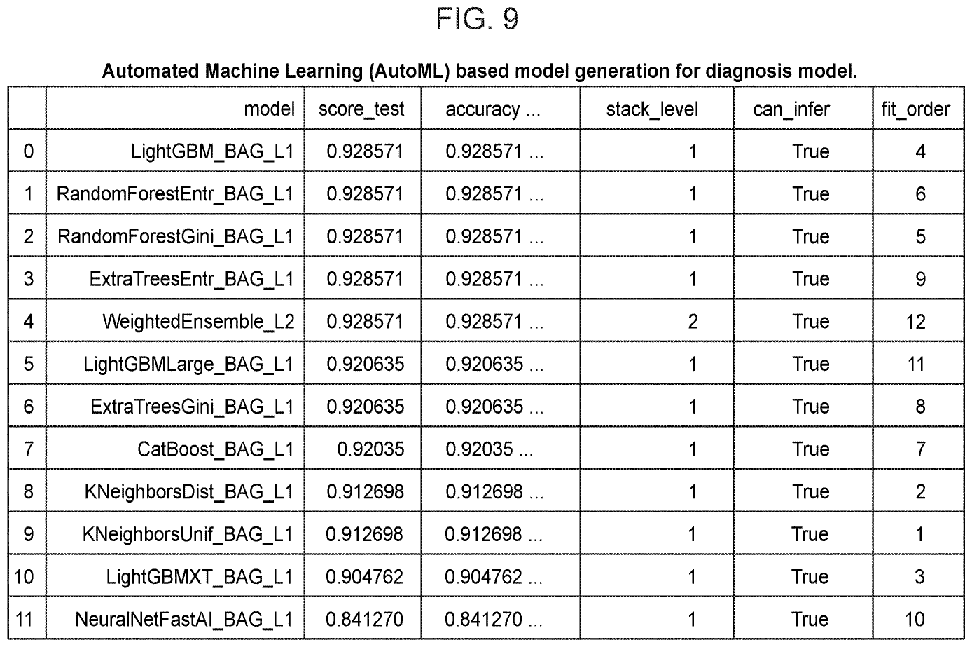

Results of applying the referral and diagnosis models are summarized in FIGS. 6A-D, FIG. 7A-D, FIG. 8A-B and FIG. 9. As described above, at steps 440 and 450, the diagnosis and referral models were applied to historical electronic health record (EHR) data from the AHP cases to determine if any of them could have potentially been diagnosed with AHP earlier. It was found that 25% of the AHP cohort could have been diagnosed earlier, on average two years prior to the date of their clinical diagnosis. Within the datasets used, many patients could not be diagnosed earlier due to the absence of EHR data in a given year, a consequence of a fragmented health system.

While this step is not shown in FIG. 4A, biochemical testing or clinical examination would next be applied to those patients, for which the diagnosis model has indicated a likelihood of an AHP diagnosis above a specified threshold, in this case above a 50% likelihood of positive AHP diagnosis.

The multi center case-control study presented in connection with FIG. 4A, aimed to develop referral and diagnosis machine learning prediction models to reduce diagnostic delays in Acute Hepatic Porphyria and apply them to identify AHP patients from a large pool of potential undiagnosed patients before any professional diagnosis of AHP. Three models were developed using multi-center EHR patient data sets; the first two models were included as referral models to predict the patient probability of a referral to the clinic (i.e., specialist), and the second combined data model was included as a diagnosis model to confirm the reference probability (i.e., diagnosis). It was found that the two-stage modeling (i.e., referral model followed by diagnosis model) approach provides a performance benefit over traditional EHR-based rare disease prediction models, in which a small number of confirmed patients are used as cases against a very large control. The current study approach provides more balanced data by combining the clinically confirmed cases and controls as cases in the reference cohort against the potential undiagnosed patients (at least one abdominal pain and age 10-65) as controls. Two models were generated in this aspect, one for each center, and the models were applied back to the controls to generate the referral probability. The diagnosis models were generated by combining the EHR data from both UCSF and UCLA with cases and controls that are clinically confirmed based on AHP disease expertise. The patients with a cut-off of ten percent referral probability are then used to infer the diagnosis model and patients with 50 percentage of probability were identified by this diagnosis model. Multiple machine learning models were developed and evaluated such as linear and tree-based baseline models, autoML-based boosted and ensemble models, and deep learning models. Among which linear and boosted tree-based models achieved the best performance, such models are useful both in terms of inference and possible future deployment as prediction tools for reference to the clinic. The models used date-restricted time-sliced EHR data and it is believed that this makes the models more robust in making the predictions. The data in case and control was restricted to only include EHR data before a potential clinical confirmation of case and control. Using feature selection it was confirmed that this approach enables the models to learn clinically relevant signs and symptoms, such as, for example, “abdominal pain,” “nausea and vomiting,” “hypertension,” “tremor,” etc. rather than concepts like “PBG,” “ALA,” “hemin,” “hepatology,” etc., which are rich in the case and control patient records.

Previous studies that used EHR data including notes for rare disease prediction generated models that lacked aspects of the present invention applied in the study presented in connection with FIG. 4A, such as clinically relevant time restriction and two-stage modeling. One previous study reported a machine learning prediction model for diagnosing AHP patients, but the applicable control for the model was the entire patient population other than the AHP cases. Further details are provided in: Cohen A M, Chamberlin S, Deloughery T, et al. Detecting rare diseases in electronic health records using machine learning and knowledge engineering: Case study of acute hepatic porphyria. PLOS ONE. 2020;15(7):e0235574. doi:10.1371/journal. pone.0235574, the disclosure of which is incorporated herein in its entirety. It is believed that such an approach may introduce noise and bias to the model at large.

Another previous study developed multiple algorithms for rare disease prediction using collaborative filtering approaches and applied them to a wide variety of rare diseases 14. Further details are provided in: Shen F, Liu S, Wang Y, Wen A, Wang L, Liu H. Utilization of Electronic Medical Records and Biomedical Literature to Support the Diagnosis of Rare Diseases Using Data Fusion and Collaborative Filtering Approaches. JMIR Medical Informatics. 2018;6(4):e11301. doi:10.2196/11301, the disclosure of which is incorporated herein in its entirety. The study focused on the performance efficiency of data fusion strategies rather than clinically relevant feature-selected disease prediction models. Other earlier reported models mostly used a similar pipeline of model generation and did not include any of the following aspects of the present invention: use of clinical notes, feature selection using external biomedical knowledge sources, data restriction on EHR data, two-stage modeling approach, evaluation using baseline, autoML and deep learning approaches.

Further details are provided in: Nemesure M D, Heinz M V, Huang R, Jacobson N C. Predictive modeling of depression and anxiety using electronic health records and a novel machine learning approach with artificial intelligence. Sci Rep. 2021; 11(1):1980. doi:10.1038/s41598-021-81368-4; Garg R, Dong S, Shah S, Jonnalagadda S R. A Bootstrap Machine Learning Approach to Identify Rare Disease Patients from Electronic Health Records. arXiv; 2016. doi:10.48550/arXiv.1609.01586; Ma F, Wang Y, Gao J, Xiao H, Zhou J. Rare disease prediction by generating quality-assured electronic health records *: 2020 SIAM International Conference on Data Mining, SDM 2020. Demeniconi C, Chawla N, eds. Proceedings of the 2020 SIAM International Conference on Data Mining, SDM 2020. Published online 2020:514-522. doi:10.1137/1.9781611976236.58; Colbaugh R, Glass K, Rudolf C, Tremblay M. Robust Ensemble Learning to Identify Rare Disease Patients from Electronic Health Records. Annu Int Conf IEEE Eng Med Biol Soc. 2018; 2018:4085-4088. doi:10.1109/EMBC.2018.8513241; Chen X, Faviez C, Vincent M, Garcelon N, Saunier S, Burgun A. Identification of Similar Patients Through Medical Concept Embedding from Electronic Health Records: A Feasibility Study for Rare Disease Diagnosis. Public Health and Informatics. Published online 2021:600-604. doi:10.3233/SHTI210241; Maguire A, Johnson M E, Denning D W, Ferreira G L C, Cassidy A. Identifying rare diseases using electronic medical records: the example of allergic bronchopulmonary aspergillosis. Pharmacoepidemiology and Drug Safety. 2017;26(7):785-791. doi:10.1002/pds.4204, the disclosures of each of which are incorporated herein in their entireties.

FIG. 4B presents probability distribution results from applying the referral and diagnosis models for UCSF and UCLA inference patients described above.

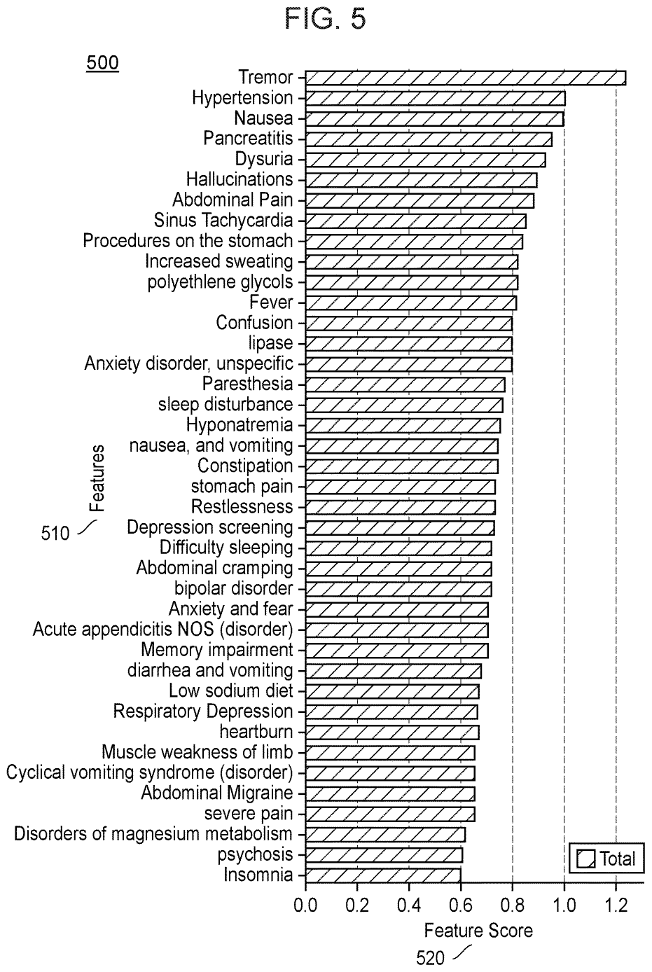

FIG. 5 presents a rank ordered list of AHP disease specific features identified by the models in process 400 discussed above. The AHP disease specific features 510 are listed on the y-axis of plot 500 and are features present in the health records obtained in connection with steps 405 and 410 of process 400. Each feature of features 510 has a feature score 520 associated with it, shown on the x-axis of plot 500. The feature score 520 associated with each disease specific feature indicates how meaningful the presence of the feature in a health record is in connection with estimating whether a referral to a specialist for AHP is warranted or whether the subject would receive a positive AHP diagnosis. Features 510 are listed from the most meaningful features at the top to the least meaningful features listed at the bottom. For example, application of the referral and diagnosis models in process 400 of FIG. 4A discovered that the presence of features tremor, hypertension or nausea in a health record are more meaningful for referral for or diagnosis of AHP than the presence of features tremor, psychosis or disorders of magnesium metabolism in a health record.

The results of training and applying the diagnosis and referral models (i.e., steps 430, 440 and 450) in connection with the application of an embodiment of FIG. 4A as well as characteristics of each dataset are set forth in FIGS. 6A-D, FIG. 7A-D, FIG. 8A-B and FIG. 9. FIG. 6A presents demographic and clinical characteristics of the case and control patients at UCSF and UCLA, used in the application presented in FIG. 4A. FIG. 6B presents performance of machine learning models for referral and diagnosis tasks in connection with the application presented in FIG. 4A. The term PPV refers to positive predictive value; the term FPR refers to false positive rate; the term FNR refers to false negative rate; the term FDR refers to false discovery rate; and the term MAE refers to mean absolute error. FIG. 6C presents performance of sequential deep learning models Recurrent Neural Network (RNN) and Convolutional Neural Network (CNN) for referral and diagnosis tasks. FIG. 6D presents expected number of a new diagnosis. FIG. 7A presents AUC for the prediction model in 5-fold CV. FIG. 7B presents class prediction error (Actual vs Predicted) where 1 indicates AHP and 0 indicates no AHP labels. FIG. 7C presents cumulative gains curve for the prediction model. FIG. 7D presents lift curve for the prediction model.

FIG. 8A presents a learning curve for prediction model. FIG. 8B presents validation curve for the prediction model. FIG. 9 presents Automated Machine Learning (AutoML) based model generation for diagnosis model.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

What is claimed is:

1. A method of training a model to estimate the presence of a disease in a subject, the method comprising:
obtaining a plurality of health records comprising health information about individuals;
modifying each health record of the plurality of health records to exclude certain health information;
identifying potential indicators of a disease based on publicly available databases, wherein the disease is a disease that occurs in approximately one in 100,000 individuals, and the potential indicators comprise health information present in the health records;
training a machine learning model to estimate the presence of the disease based on the potential indicators of the disease using a first subset of the modified health records;
evaluating the accuracy of estimates of the presence of the disease based on the potential indicators of the disease made by the machine learning model using a second subset of the modified health records; and
further training the machine learning model using the plurality of health records and based at least in part on the results of evaluating the accuracy of the machine learning model.

2. A method of inferring the presence of a disease in a subject, the method comprising:
obtaining a health record comprising health information about a subject;
applying a model trained according to the method of claim 1 to the health record of the subject to estimate the presence in a disease in the subject; and
estimating the presence of the disease in the subject based on the results of applying the model.

3. A method of clinically evaluating the presence of a disease in a subject, the method comprising:
estimating the presence of a disease in a subject according to the method of claim 2, wherein the estimate of the presence of the disease comprises a confidence score; and
clinically evaluating the presence of the disease in the subject, in the event the confidence score exceeds a predetermined threshold.

4. A method of estimating the presence of a disease in a plurality of subjects, the method comprising:
obtaining health records comprising health information about a plurality of subjects;
applying a model trained according to the method of claim 1 to the health records to estimate the presence in a disease in each subject of the plurality of subjects; and
inferring the presence of the disease in each subject of the plurality of subjects based on the results of applying the model.

5. A method of clinically evaluating the presence of a disease in a plurality of subjects, the method comprising:
estimating the presence of a disease in a plurality of subjects according to the method of claim 4, wherein each estimate of the presence of the disease in each subject of the plurality of subjects comprises a confidence score; and
clinically evaluating the presence of the disease in each subject, in the event the confidence score for such subject exceeds a predetermined threshold.

6. A method of training a model to recommend a referral for evaluating the presence of a disease in a subject, the method comprising:
obtaining a plurality of health records comprising health information about individuals;
modifying each health record of the plurality of health records to exclude certain health information;
identifying potential indicators of a disease based on publicly available databases, wherein the disease is a disease that occurs in approximately one in 100,000 individuals, and the potential indicators comprise health information present in the health records;
training a machine learning model to recommend a referral for evaluating the presence of a disease based on the potential indicators of the disease using a first subset of the modified health records;
evaluate the accuracy of the machine learning model based on the potential indicators of the disease using a second subset of the modified health records; and
further train the machine learning model using the plurality of health records and based at least in part on the results of evaluating the accuracy of the machine learning model.

7. A method of recommending a referral for evaluating the presence of a disease in a subject, the method comprising:
obtaining a health record comprising health information about a subject; applying a model trained according to the method of claim 6 to the health record of the subject to recommend a referral for evaluating the presence of a disease in the subject; and
recommending a referral for evaluating the presence of a disease in the subject based on the results of applying the model.

8. A method of estimating the presence of a disease in a subject, the method comprising:
determining whether a referral for evaluating the presence of a disease in a subject is recommended according to the method of claim 7, wherein the recommendation for a referral comprises a confidence score; and
applying a model trained according to the method of claim 1 to a health record of the subject to estimate the presence in a disease in the subject, in the event the confidence score exceeds a predetermined threshold; and
estimating the presence of the disease in the subject based on the results of applying the model.

9. A method of clinically evaluating the presence of a disease in a subject, the method comprising:
estimating the presence of a disease in a subject according to the method of claim 8, wherein the estimate of the presence of the disease comprises a confidence score; and
clinically evaluating the presence of the disease, in the event the confidence score exceeds a predetermined threshold.

10. A method of recommending a referral for evaluating the presence of a disease in a plurality of subjects, the method comprising:
obtaining health records comprising health information about a plurality of subjects;
applying a model trained according to the method of claim 6 to the health records to recommend a referral for evaluating the presence of a disease in each subject of the plurality of subjects; and
recommending a referral for evaluating the presence of a disease in each subject of the plurality of subjects based on the results of applying the model.

11. A method of estimating the presence of a disease in a plurality of subjects, the method comprising:
determining whether a referral for evaluating the presence of a disease in each subject of a plurality of subjects is recommended according to the method of claim 10, wherein the recommendation for a referral comprises a confidence score;
applying a model trained according to the method of claim 1 to health records of each subject of the plurality of subjects to estimate the presence of a disease of each subject of the plurality of subjects, in the event the confidence score exceeds a predetermined threshold; and
estimating the presence of the disease in each subject of the plurality of subjects based on the results of applying the model.

12. A method of clinically evaluating the presence of a disease in a plurality of subjects, the method comprising:
estimating the presence of a disease in a plurality of subjects according to the method of claim 11, wherein each estimate of the presence of the disease comprises a confidence score; and
clinically evaluating the presence of the disease for each subject of the plurality of subjects, in the event the confidence score exceeds a predetermined threshold.

13. The method according to any of the previous claims, wherein the disease is acute hepatic porphyria.

14. The method according to any of the previous claims, wherein the health records comprise structured electronic health records.

15. The method according to any of the previous claims, wherein the health records comprise structured and unstructured electronic health records.

16. The method according to any of the previous claims, wherein the health records originate from a single medical center.

17. The method according to any of claims 1 to 15, wherein the health records originate from more than one medical center.

18. The method according to any of the previous claims, wherein the health information comprises one or more of: a diagnosis, information about a procedure, laboratory results, medication information, vitals information, family history or demographic information.

19. The method according to any of the previous claims, wherein modifying each health record of the plurality of health records to exclude certain health information comprises excluding health information subsequent to an occurrence of a clinically relevant event.

20. The method according to claim 19, wherein the occurrence of a clinically relevant event comprises one or more of: an assignment of a disease code or negative confirmation of the disease or a first visit to a specialist clinic.

21. The method according to any of the previous claims, wherein potential indicators of the disease comprise one or more of: tremor, hypertension, nausea, pancreatitis, dysuria, hallucinations, abdominal pain, sinus tachycardia, procedures on the stomach or increased sweating.

22. The method according to any of the previous claims, wherein training a machine learning model to estimate the presence of the disease based on the potential indicators of the disease using a first subset of the modified health records comprises utilizing one or more of stratified sampling, held out test set, cross validation, hyper parameter tuning or random grid search.

23. The method according to any of the previous claims, wherein evaluating the accuracy of estimates of the presence of the disease comprises using one or more of held out test sets and cross validation evaluations.

24. The method according to any of the previous claims, wherein further training the machine learning model using the plurality of health records comprises training the machine learning model using the plurality of health records in their entirety.

25. The method according to any of the previous claims, wherein clinically evaluating the presence of the disease comprises clinical evaluation of the subject by a specialist.

26. The method according to any of the previous claims, wherein clinically evaluating the presence of the disease comprises conducting biochemical analysis.

27. The method according to claim 26, wherein the biochemical analysis comprises one or more of identifying urinary and plasma amniolevulinic acid levels or identifying porphobilinogen (PBG) levels.

28. The method according to any of the previous claims, wherein the publicly available databases comprise one or more of a rare disease database or a semantic predictions database.

29. The method according to any of the previous claims, wherein the subject belongs to an at-risk population for the disease.

30. The method according to claim 29, wherein the at-risk population comprises subjects exhibiting specified symptoms.

31. The method according to claim 30, wherein the specified symptoms comprise abdominal pain over a three-year period.

32. The method according to claim 3, wherein the predetermined threshold is 50%.

33. The method according to any of claims 6 to 12, wherein the machine learning model comprises a first referral model and a second referral model.

34. The method according to any of claims 5 or 9, wherein the predetermined threshold is 50%.

35. The method according to any of claims 8 or 11, wherein the predetermined threshold is 10%.

36. The method according to any of the previous claims, wherein the machine learning model comprises one or more of: a statistical model, a linear model, a computational model, a tree-based model, a convolutional neural network, an artificial neural network or a deep learning network.

37. The method according to any of the previous claims, wherein training the machine learning model comprises one or more of: an unsupervised learning technique, a semi-supervised learning technique or a supervised learning technique.

38. The method according to any of the previous claims, wherein the disease is one of: factor XIII deficiency, Hutchinson-Gilford progeria syndrome, Barth syndrome (BTHS), nephrogenic diabetes insipidus, congenital generalized lipodystrophy (also known as Berardinelli-Seip lipodystrophy), fibrolamellar hepatocellular carcinoma (FHCC), autoimmune polyendocrine syndrome type 1 (APS-1), cerebral creatine deficiency syndromes, cyclic neutropenia, goblet cell carcinoid, ablepharon-macrostomia syndrome (AMS), Alexander disease, Baller-Gerold syndrome (BGS), Bernard-Soulier syndrome (BSS), Caroli disease, Cleidocranial dysostosis (CCD), cold agglutinin disease (CAD), congenital afibrinogenemia, cutaneous T cell lymphoma (CTCL), cutis laxa, factor XI or plasma thromboplastin antecedent, factor XII deficiency, familial partial lipodystrophy, fatal insomnia, glycogen storage disease type VI (GSD VI), hypothalamic disease, keratosis follicularis spinulosa decalvans, Miller syndrome, opsoclonus myoclonus syndrome (OMS), paroxysmal nocturnal hemoglobinuria (PNH) or Crigler-Najjar syndrome.

39. The method according to any of the previous claims, wherein the subject is human.

40. The method according to any of the previous claims, wherein the method is a computer-implemented method.

41. A system for training a model to estimate the presence of a disease in a subject comprising:
a processor comprising memory operably coupled to the processor, wherein the memory comprises instructions stored thereon, which, when executed by the processor, cause the processor to:
obtain a plurality of health records comprising health information about individuals;
modify each health record of the plurality of health records to exclude certain health information;
identify potential indicators of a disease based on publicly available databases, wherein the disease is a disease that occurs in approximately one in 100,000 individuals, and the potential indicators comprise health information present in the health records;
train a machine learning model to estimate the presence of the disease based on the potential indicators of the disease using a first subset of the modified health records;
evaluate the accuracy of estimates of the presence of the disease based on the potential indicators of the disease made by the machine learning model using a second subset of the modified health records; and
further train the machine learning model using the plurality of health records and based at least in part on the results of evaluating the accuracy of the machine learning model.

42. The system according to claim 41, wherein the memory further comprises instructions stored thereon, which, when executed by the processor, cause the processor to:
obtain a health record comprising health information about a subject;
apply the trained model to the health record of the subject to estimate the presence in a disease in the subject; and
estimate the presence of the disease in the subject based on the results of applying the model.

43. The system according to claim 42, wherein the memory further comprises instructions stored thereon, which, when executed by the processor, cause the processor to:
estimate the presence of a disease in the subject, wherein the estimate of the presence of the disease comprises a confidence score; and
indicate a determination to clinically evaluate the presence of the disease in the subject, in the event the confidence score exceeds a predetermined threshold.

44. The system according to claim 41, wherein the memory further comprises instructions stored thereon, which, when executed by the processor, cause the processor to:
obtain health records comprising health information about a plurality of subjects;
apply the trained model to the health records to estimate the presence of a disease in each subject of the plurality of subjects; and
infer the presence of the disease in each subject of the plurality of subjects based on the results of applying the model.

45. The system according to claim 44, wherein the memory further comprises instructions stored thereon, which, when executed by the processor, cause the processor to:
estimate the presence of a disease in a plurality of subjects, wherein each estimate of the presence of the disease in each subject of the plurality of subjects comprises a confidence score; and
indicate a determination to clinically evaluate the presence of the disease in each subject, in the event the confidence score for such subject exceeds a predetermined threshold.

46. A system for training a model to recommend a referral for evaluating the presence of a disease in a subject, the system comprising:
a processor comprising memory operably coupled to the processor, wherein the memory comprises instructions stored thereon, which, when executed by the processor, cause the processor to:
obtain a plurality of health records comprising health information about individuals;
modify each health record of the plurality of health records to exclude certain health information;
identify potential indicators of a disease based on publicly available databases, wherein the disease is a disease that occurs in approximately one in 100,000 individuals, and the potential indicators comprise health information present in the health records;
train a machine learning model to recommend a referral for evaluating the presence of a disease based on the potential indicators of the disease using a first subset of the modified health records;
evaluate the accuracy of the machine learning model based on the potential indicators of the disease using a second subset of the modified health records; and
further train the machine learning model using the plurality of health records and based at least in part on the results of evaluating the accuracy of the machine learning model.

47. The system according to claim 46, wherein the memory further comprises instructions stored thereon, which, when executed by the processor, cause the processor to:
obtain a health record comprising health information about a subject;
apply the trained model to the health record of the subject to recommend a referral for evaluating the presence of a disease in the subject; and
recommend a referral for evaluating the presence of a disease in the subject based on the results of applying the model.

48. The system according to claim 47, wherein the memory further comprises instructions stored thereon, which, when executed by the processor, cause the processor to:
determine whether a referral for evaluating the presence of a disease in a subject is recommended, wherein the recommendation for a referral comprises a confidence score and wherein the model comprises a referral model;
obtain a plurality of health records comprising health information about individuals;
modify each health record of the plurality of health records to exclude certain health information;
identify potential indicators of a disease based on publicly available databases, wherein the disease is a disease that occurs in approximately one in 100,000 individuals, and the potential indicators comprise health information present in the health records;
train a machine learning diagnosis model to estimate the presence of the disease based on the potential indicators of the disease using a first subset of the modified health records;
evaluate the accuracy of estimates of the presence of the disease based on the potential indicators of the disease made by the machine learning diagnosis model using a second subset of the modified health records;
further train the machine learning diagnosis model using the plurality of health records and based at least in part on the results of evaluating the accuracy of the machine learning model;
apply the trained diagnosis model to a health record of the subject to estimate the presence in a disease in the subject, in the event the confidence score generated by the referral model exceeds a predetermined threshold; and
estimate the presence of the disease in the subject based on the results of applying the diagnosis model.

49. The system according to claim 48, wherein the memory further comprises instructions stored thereon, which, when executed by the processor, cause the processor to:
estimate the presence of a disease in a subject using the diagnosis model, wherein the estimate of the presence of the disease comprises a confidence score; and
indicate a determination to clinically evaluate the presence of the disease, in the event the confidence score exceeds a predetermined threshold.

50. The system according to claim 46, wherein the memory further comprises instructions stored thereon, which, when executed by the processor, cause the processor to:
obtain health records comprising health information about a plurality of subjects;
apply the trained model to the health records to recommend a referral for evaluating the presence of a disease in each subject of the plurality of subjects; and
recommend a referral for evaluating the presence of a disease in each subject of the plurality of subjects based on the results of applying the model.

51. The system according to claim 50, wherein the memory further comprises instructions stored thereon, which, when executed by the processor, cause the processor to:
determine whether a referral for evaluating the presence of a disease in each subject of a plurality of subjects is recommended, wherein the recommendation for a referral comprises a confidence score and wherein the model is a referral model;
obtain a plurality of health records comprising health information about individuals;
modify each health record of the plurality of health records to exclude certain health information;
identify potential indicators of a disease based on publicly available databases, wherein the disease is a disease that occurs in approximately one in 100,000 individuals, and the potential indicators comprise health information present in the health records;
train a machine learning diagnosis model to estimate the presence of the disease based on the potential indicators of the disease using a first subset of the modified health records;
evaluate the accuracy of estimates of the presence of the disease based on the potential indicators of the disease made by the machine learning diagnosis model using a second subset of the modified health records;
further train the machine learning diagnosis model using the plurality of health records and based at least in part on the results of evaluating the accuracy of the machine learning model;
apply the diagnosis model to health records of each subject of the plurality of subjects to estimate the presence of a disease of each subject of the plurality of subjects, in the event the confidence score generated by the referral model exceeds a predetermined threshold; and
estimate the presence of the disease in each subject of the plurality of subjects based on the results of applying the model.

52. The system according to claim 51, wherein the memory further comprises instructions stored thereon, which, when executed by the processor, cause the processor to:
estimate the presence of a disease in a plurality of subjects, wherein each estimate of the presence of the disease comprises a confidence score; and
indicate a determination to clinically evaluate the presence of the disease for each subject of the plurality of subjects, in the event the confidence score exceeds a predetermined threshold.

53. The system according to any of claims 41 to 52, wherein the disease is acute hepatic porphyria.

54. The system according to any of claims 41 to 53, wherein the health records comprise structured electronic health records.

55. The system according to any of claims 41 to 54, wherein the health records comprise structured and unstructured electronic health records.

56. The system according to any of claims 41 to 55, wherein the health records originate from a single medical center.

57. The system according to any of claims 41 to 55, wherein the health records originate from more than one medical center.

58. The system according to any of claims 41 to 57, wherein the health information comprises one or more of: a diagnosis, information about a procedure, laboratory results, medication information, vitals information, family history or demographic information.

59. The system according to any of claims 41 to 58, wherein modifying each health record of the plurality of health records to exclude certain health information comprises excluding health information subsequent to an occurrence of a clinically relevant event.

60. The system according to claim 59, wherein the occurrence of a clinically relevant event comprises one or more of: an assignment of a disease code or negative confirmation of the disease or a first visit to a specialist clinic.

61. The system according to any of claims 41 to 60, wherein potential indicators of the disease comprise one or more of: tremor, hypertension, nausea, pancreatitis, dysuria, hallucinations, abdominal pain, sinus tachycardia, procedures on the stomach or increased sweating.

62. The system according to any of claims 41 to 61, wherein training a machine learning model to estimate the presence of the disease based on the potential indicators of the disease using a first subset of the modified health records comprises utilizing one or more of stratified sampling, held out test set, cross validation, hyper parameter tuning or random grid search.

63. The system according to any of claims 41 to 62, wherein evaluating the accuracy of estimates of the presence of the disease comprises using one or more of held out test sets and cross validation evaluations.

64. The system according to any of claims 41 to 63, wherein further training the machine learning model using the plurality of health records comprises training the machine learning model using the plurality of health records in their entirety.

65. The system according to any of claims 41 to 64, wherein clinically evaluating the presence of the disease comprises clinical evaluation of the subject by a specialist.

66. The system according to any of claims 41 to 65, wherein clinically evaluating the presence of the disease comprises conducting biochemical analysis.

67. The system according to claim 66, wherein the biochemical analysis comprises one or more of identifying urinary and plasma amniolevulinic acid levels or identifying porphobilinogen (PBG) levels.

68. The system according to any of claims 41 to 67, wherein the publicly available databases comprise one or more of a rare disease database or a semantic predictions database.

69. The system according to any of claims 41 to 68, wherein the subject belongs to an at-risk population for the disease.

70. The system according to claim 69, wherein the at-risk population comprises subjects exhibiting specified symptoms.

71. The system according to claim 70, wherein the specified symptoms comprise abdominal pain over a three-year period.

72. The system according to claim 43, wherein the predetermined threshold is 50%.

73. The system according to any of claims 46 to 52, wherein the machine learning model comprises a first referral model and a second referral model.

74. The system according to any of claims 45 or 49, wherein the predetermined threshold is 50%.

75. The system according to any of claims 48 or 51, wherein the predetermined threshold is 10%.

76. The system according to any of claims 41 to 75, wherein the machine learning model comprises one or more of: a statistical model, a linear model, a computational model, a tree-based model, a convolutional neural network, an artificial neural network or a deep learning network.

77. The system according to any of claims 41 to 76, wherein training the machine learning model comprises one or more of: an unsupervised learning technique, a semi-supervised learning technique or a supervised learning technique.

78. The system according to any of claims 41 to 77, wherein the disease is one of: factor XIII deficiency, Hutchinson-Gilford progeria syndrome, Barth syndrome (BTHS), nephrogenic diabetes insipidus, congenital generalized lipodystrophy (also known as Berardinelli-Seip lipodystrophy), fibrolamellar hepatocellular carcinoma (FHCC), autoimmune polyendocrine syndrome type 1 (APS-1), cerebral creatine deficiency syndromes, cyclic neutropenia, goblet cell carcinoid, ablepharon-macrostomia syndrome (AMS), Alexander disease, Baller-Gerold syndrome (BGS), Bernard-Soulier syndrome (BSS), Caroli disease, Cleidocranial dysostosis (CCD), cold agglutinin disease (CAD), congenital afibrinogenemia, cutaneous T cell lymphoma (CTCL), cutis laxa, factor XI or plasma thromboplastin antecedent, factor XII deficiency, familial partial lipodystrophy, fatal insomnia, glycogen storage disease type VI (GSD VI), hypothalamic disease, keratosis follicularis spinulosa decalvans, Miller syndrome, opsoclonus myoclonus syndrome (OMS), paroxysmal nocturnal hemoglobinuria (PNH) or Crigler-Najjar syndrome.

79. The system according to any of claims 41 to 78, wherein the subject is human.

80. A non-transitory computer readable storage medium comprising instructions stored thereon, the instructions comprising:
algorithm for obtaining a plurality of health records comprising health information about individuals;
algorithm for modifying each health record of the plurality of health records to exclude certain health information;
algorithm for identifying potential indicators of a disease based on publicly available databases, wherein the disease is a disease that occurs in approximately one in 100,000 individuals, and the potential indicators comprise health information present in the health records;
algorithm for training a machine learning model to estimate the presence of the disease based on the potential indicators of the disease using a first subset of the modified health records;
algorithm for evaluating the accuracy of estimates of the presence of the disease based on the potential indicators of the disease made by the machine learning model using a second subset of the modified health records; and
algorithm for further training the machine learning model using the plurality of health records and based at least in part on the results of evaluating the accuracy of the machine learning model.

81. The non-transitory computer readable storage medium of claim 80, the instructions store thereon further comprising:
algorithm for obtaining a health record comprising health information about a subject;
algorithm for applying the trained model to the health record of the subject to estimate the presence in a disease in the subject; and
algorithm for estimating the presence of the disease in the subject based on the results of applying the model.

82. The non-transitory computer readable storage medium of claim 81, the instructions store thereon further comprising:
algorithm for estimating the presence of a disease in a subject, wherein the estimate of the presence of the disease comprises a confidence score; and
algorithm for indicating a determination to clinically evaluate the presence of the disease in the subject, in the event the confidence score exceeds a predetermined threshold.

83. The non-transitory computer readable storage medium of claim 80, the instructions store thereon further comprising:
algorithm for obtaining health records comprising health information about a plurality of subjects;
algorithm for applying the trained model to the health records to estimate the presence in a disease in each subject of the plurality of subjects; and
algorithm for inferring the presence of the disease in each subject of the plurality of subjects based on the results of applying the model.

84. The non-transitory computer readable storage medium of claim 83, the instructions store thereon further comprising:
algorithm for estimating the presence of a disease in a plurality of subjects, wherein each estimate of the presence of the disease in each subject of the plurality of subjects comprises a confidence score; and
algorithm for indicating a determination to clinically evaluate the presence of the disease in each subject, in the event the confidence score for such subject exceeds a predetermined threshold.

85. A non-transitory computer readable storage medium comprising instructions stored thereon, the instructions comprising:
algorithm for obtaining a plurality of health records comprising health information about individuals;
algorithm for modifying each health record of the plurality of health records to exclude certain health information;
algorithm for identifying potential indicators of a disease based on publicly available databases, wherein the disease is a disease that occurs in approximately one in 100,000 individuals, and the potential indicators comprise health information present in the health records;
algorithm for training a machine learning model to recommend a referral for evaluating the presence of a disease based on the potential indicators of the disease using a first subset of the modified health records;
algorithm for evaluating the accuracy of the machine learning model based on the potential indicators of the disease using a second subset of the modified health records; and
algorithm for further training the machine learning model using the plurality of health records and based at least in part on the results of evaluating the accuracy of the machine learning model.

86. The non-transitory computer readable storage medium of claim 85, the instructions store thereon further comprising:
algorithm for obtaining a health record comprising health information about a subject;
algorithm for applying the trained model to the health record of the subject to recommend a referral for evaluating the presence of a disease in the subject;
and algorithm for recommending a referral for evaluating the presence of a disease in the subject based on the results of applying the model.

87. The non-transitory computer readable storage medium of claim 86, the instructions store thereon further comprising:
algorithm for determining whether a referral for evaluating the presence of a disease in a subject is recommended, wherein the recommendation for a referral comprises a confidence score and wherein the model is a referral model;
algorithm for obtaining a plurality of health records comprising health information about individuals;
algorithm for modifying each health record of the plurality of health records to exclude certain health information;
algorithm for identifying potential indicators of a disease based on publicly available databases, wherein the disease is a disease that occurs in approximately one in 100,000 individuals, and the potential indicators comprise health information present in the health records;
algorithm for training a machine learning diagnosis model to estimate the presence of the disease based on the potential indicators of the disease using a first subset of the modified health records;
algorithm for evaluating the accuracy of estimates of the presence of the disease based on the potential indicators of the disease made by the machine learning diagnosis model using a second subset of the modified health records;
algorithm for further training the machine learning diagnosis model using the plurality of health records and based at least in part on the results of evaluating the accuracy of the machine learning model;
algorithm for applying the diagnosis model to a health record of the subject to estimate the presence in a disease in the subject, in the event the confidence score generated by the referral model exceeds a predetermined threshold; and
algorithm for estimating the presence of the disease in the subject based on the results of applying the diagnosis model.

88. The non-transitory computer readable storage medium of claim 87, the instructions store thereon further comprising:
algorithm for estimating the presence of a disease in a subject, wherein the estimate of the presence of the disease comprises a confidence score; and
algorithm for indicating a determination to clinically evaluate the presence of the disease, in the event the confidence score exceeds a predetermined threshold.

89. The non-transitory computer readable storage medium of claim 85, the instructions store thereon further comprising:
algorithm for obtaining health records comprising health information about a plurality of subjects;
algorithm for applying the trained model to the health records to recommend a referral for evaluating the presence of a disease in each subject of the plurality of subjects; and
algorithm for recommending a referral for evaluating the presence of a disease in each subject of the plurality of subjects based on the results of applying the model.

90. The non-transitory computer readable storage medium of claim 89, the instructions store thereon further comprising:
algorithm for determining whether a referral for evaluating the presence of a disease in each subject of a plurality of subjects is recommended, wherein the recommendation for a referral comprises a confidence score and wherein the model is a referral model;
algorithm for obtaining a plurality of health records comprising health information about individuals;
algorithm for modifying each health record of the plurality of health records to exclude certain health information;
algorithm for identifying potential indicators of a disease based on publicly available databases, wherein the disease is a disease that occurs in approximately one in 100,000 individuals, and the potential indicators comprise health information present in the health records;
algorithm for training a machine learning diagnosis model to estimate the presence of the disease based on the potential indicators of the disease using a first subset of the modified health records;
algorithm for evaluating the accuracy of estimates of the presence of the disease based on the potential indicators of the disease made by the machine learning diagnosis model using a second subset of the modified health records;
algorithm for further training the machine learning diagnosis model using the plurality of health records and based at least in part on the results of evaluating the accuracy of the machine learning model;
algorithm for applying the trained diagnosis model to health records of each subject of the plurality of subjects to estimate the presence of a disease of each subject of the plurality of subjects, in the event the confidence score generated by the referral model exceeds a predetermined threshold; and
algorithm for estimating the presence of the disease in each subject of the plurality of subjects based on the results of applying the model.

91. The non-transitory computer readable storage medium of claim 90, the instructions store thereon further comprising:
algorithm for estimating the presence of a disease in a plurality of subjects, wherein each estimate of the presence of the disease comprises a confidence score; and
algorithm for indicating a determination to clinically evaluate the presence of the disease for each subject of the plurality of subjects, in the event the confidence score exceeds a predetermined threshold.

92. The non-transitory computer readable storage medium according to any of claims 80 to 91, wherein the disease is acute hepatic porphyria.

93. The non-transitory computer readable storage medium according to any of claims 80 to 92, wherein the health records comprise structured electronic health records.

94. The non-transitory computer readable storage medium according to any of claims 80 to 93, wherein the health records comprise structured and unstructured electronic health records.

95. The non-transitory computer readable storage medium according to any of claims 80 to 94, wherein the health records originate from a single medical center.

96. The non-transitory computer readable storage medium according to any of claims 80 to 94, wherein the health records originate from more than one medical center.

97. The non-transitory computer readable storage medium according to any of claims 80 to 96, wherein the health information comprises one or more of: a diagnosis, information about a procedure, laboratory results, medication information, vitals information, family history or demographic information.

98. The non-transitory computer readable storage medium according to any of claims 80 to 98, wherein modifying each health record of the plurality of health records to exclude certain health information comprises excluding health information subsequent to an occurrence of a clinically relevant event.

99. The non-transitory computer readable storage medium according to claim 98, wherein the occurrence of a clinically relevant event comprises one or more of: an assignment of a disease code or negative confirmation of the disease or a first visit to a specialist clinic.

100. The non-transitory computer readable storage medium according to any of claims 80 to 99, wherein potential indicators of the disease comprise one or more of: tremor, hypertension, nausea, pancreatitis, dysuria, hallucinations, abdominal pain, sinus tachycardia, procedures on the stomach or increased sweating.

101. The non-transitory computer readable storage medium according to any of claims 80 to 100, wherein training a machine learning model to estimate the presence of the disease based on the potential indicators of the disease using a first subset of the modified health records comprises utilizing one or more of stratified sampling, held out test set, cross validation, hyper parameter tuning or random grid search.

102. The non-transitory computer readable storage medium according to any of claims 80 to 101, wherein evaluating the accuracy of estimates of the presence of the disease comprises using one or more of held out test sets and cross validation evaluations.

103. The non-transitory computer readable storage medium according to any of claims 80 to 103, wherein further training the machine learning model using the plurality of health records comprises training the machine learning model using the plurality of health records in their entirety.

104. The non-transitory computer readable storage medium according to any of claims 80 to 103, wherein clinically evaluating the presence of the disease comprises clinical evaluation of the subject by a specialist.

105. The non-transitory computer readable storage medium according to any of claims 80 to 104, wherein clinically evaluating the presence of the disease comprises conducting biochemical analysis.

106. The non-transitory computer readable storage medium according to claim 105, wherein the biochemical analysis comprises one or more of identifying urinary and plasma amniolevulinic acid levels or identifying porphobilinogen (PBG) levels.

107. The non-transitory computer readable storage medium according to any of claims 80 to 106, wherein the publicly available databases comprise one or more of a rare disease database or a semantic predictions database.

108. The non-transitory computer readable storage medium according to any of claims 80 to 107, wherein the subject belongs to an at-risk population for the disease.

109. The non-transitory computer readable storage medium according to claim 108, wherein the at-risk population comprises subjects exhibiting specified symptoms.

110. The non-transitory computer readable storage medium according to claim 109, wherein the specified symptoms comprise abdominal pain over a three-year period.

111. The non-transitory computer readable storage medium according to claim 82, wherein the predetermined threshold is 50%.

112. The non-transitory computer readable storage medium according to any of claims 85 to 91, wherein the machine learning model comprises a first referral model and a second referral model.

113. The non-transitory computer readable storage medium according to any of claims 84 or 18, wherein the predetermined threshold is 50%.

114. The non-transitory computer readable storage medium according to any of claims 17 or 20, wherein the predetermined threshold is 10%.

115. The non-transitory computer readable storage medium according to any of claims 80 to 114, wherein the machine learning model comprises one or more of:
a statistical model, a linear model, a computational model, a tree-based model, a convolutional neural network, an artificial neural network or a deep learning network.

116. The non-transitory computer readable storage medium according to any of claims 80 to 115, wherein training the machine learning model comprises one or more of: an unsupervised learning technique, a semi-supervised learning technique or a supervised learning technique.

117. The non-transitory computer readable storage medium according to any of claims 80 to 116, wherein the disease is one of: factor XIII deficiency, Hutchinson-Gilford progeria syndrome, Barth syndrome (BTHS), nephrogenic diabetes insipidus, congenital generalized lipodystrophy (also known as Berardinelli-Seip lipodystrophy), fibrolamellar hepatocellular carcinoma (FHCC), autoimmune polyendocrine syndrome type 1 (APS-1), cerebral creatine deficiency syndromes, cyclic neutropenia, goblet cell carcinoid, ablepharon-macrostomia syndrome (AMS), Alexander disease, Baller-Gerold syndrome (BGS), Bernard-Soulier syndrome (BSS), Caroli disease, Cleidocranial dysostosis (CCD), cold agglutinin disease (CAD), congenital afibrinogenemia, cutaneous T cell lymphoma (CTCL), cutis laxa, factor XI or plasma thromboplastin antecedent, factor XII deficiency, familial partial lipodystrophy, fatal insomnia, glycogen storage disease type VI (GSD VI), hypothalamic disease, keratosis follicularis spinulosa decalvans, Miller syndrome, opsoclonus myoclonus syndrome (OMS), paroxysmal nocturnal hemoglobinuria (PNH) or Crigler-Najjar syndrome.

118. The non-transitory computer readable storage medium according to any of claims 80 to 117, wherein the subject is human.
